SEQUENCE LISTING (67 sequences)

SEQ ID NO: 1 - protein, 38 aa, Homo sapiens
DAEFRHDSGYEVHHQKLVFFAEDVGSNKGAIIGLMVGG

SEQ ID NO: 2 - protein, 39 aa, Homo sapiens
DAEFRHDSGYEVHHQKLVFFAEDVGSNKGAIIGLMVGGV

SEQ ID NO: 3 - protein, 40 aa, Homo sapiens
DAEFRHDSGYEVHHQKLVFFAEDVGSNKGAIIGLMVGGVV

SEQ ID NO: 4 - protein, 41 aa, Homo sapiens
DAEFRHDSGYEVHHQKLVFFAEDVGSNKGAIIGLMVGGVVI

SEQ ID NO: 5 - protein, 42 aa, Homo sapiens [sequence omitted]

SEQ ID NO: 6 - protein, 43 aa, Homo sapiens
DAEFRHDSGYEVHHQKLVFFAEDVGSNKGAIIGLMVGGVVIAT

SEQ ID NO: 7 - protein, 37 aa, Homo sapiens
AEFRHDSGYEVHHQKLVFFAEDVGSNKGAIIGLMVGG

SEQ ID NO: 8 - protein, 38 aa, Homo sapiens
AEFRHDSGYEVHHQKLVFFAEDVGSNKGAIIGLMVGGV

SEQ ID NO: 9 - protein, 39 aa, Homo sapiens
AEFRHDSGYEVHHQKLVFFAEDVGSNKGAIIGLMVGGVV

SEQ ID NO: 10 - protein, 40 aa, Homo sapiens
AEFRHDSGYEVHHQKLVFFAEDVGSNKGAIIGLMVGGVVI

SEQ ID NO: 11 - protein, 41 aa, Homo sapiens
AEFRHDSGYEVHHQKLVFFAEDVGSNKGAIIGLMVGGVVIA

SEQ ID NO: 12 - protein, 42 aa, Homo sapiens
AEFRHDSGYEVHHQKLVFFAEDVGSNKGAIIGLMVGGVVIAT

SEQ ID NO: 13 - protein, 36 aa, Homo sapiens
EFRHDSGYEVHHQKLVFFAEDVGSNKGAIIGLMVGG

SEQ ID NO: 14 - protein, 37 aa, Homo sapiens
EFRHDSGYEVHHQKLVFFAEDVGSNKGAIIGLMVGGV

SEQ ID NO: 15 - protein, 38 aa, Homo sapiens
EFRHDSGYEVHHQKLVFFAEDVGSNKGAIIGLMVGGVV

SEQ ID NO: 16 - protein, 39 aa, Homo sapiens
EFRHDSGYEVHHQKLVFFAEDVGSNKGAIIGLMVGGVVI

SEQ ID NO: 17 - protein, 40 aa, Homo sapiens
EFRHDSGYEVHHQKLVFFAEDVGSNKGAIIGLMVGGVVIA

SEQ ID NO: 18 - protein, 41 aa, Homo sapiens
EFRHDSGYEVHHQKLVFFAEDVGSNKGAIIGLMVGGVVIAT

SEQ ID NO: 19 - protein, 28 aa, Homo sapiens
EVHHQKLVFFAEDVGSNKGAIIGLMVGG

SEQ ID NO: 20 - protein, 29 aa, Homo sapiens
EVHHQKLVFFAEDVGSNKGAIIGLMVGGV

SEQ ID NO: 21 - protein, 30 aa, Homo sapiens
EVHHQKLVFFAEDVGSNKGAIIGLMVGGVV

SEQ ID NO: 22 - protein, 31 aa, Homo sapiens
EVHHQKLVFFAEDVGSNKGAIIGLMVGGVVI

SEQ ID NO: 23 - protein, 32 aa, Homo sapiens
EVHHQKLVFFAEDVGSNKGAIIGLMVGGVVIA

SEQ ID NO: 24 - protein, 33 aa, Homo sapiens
EVHHQKLVFFAEDVGSNKGAIIGLMVGGVVIAT

SEQ ID NO: 25 - protein, 24 aa, Homo sapiens
QKLVFFAEDVGSNKGAIIGLMVGG

SEQ ID NO: 26 - protein, 36 aa, Homo sapiens; identical to SEQ ID NO: 13
  MOD_RES (1)..(1): PYRROLIDONE CARBOXYLIC ACID

SEQ ID NO: 27 - protein, 37 aa, Homo sapiens; identical to SEQ ID NO: 14
  MOD_RES (1)..(1): PYRROLIDONE CARBOXYLIC ACID

SEQ ID NO: 28 - protein, 38 aa, Homo sapiens; identical to SEQ ID NO: 15
  MOD_RES (1)..(1): PYRROLIDONE CARBOXYLIC ACID

SEQ ID NO: 29 - protein, 39 aa, Homo sapiens; identical to SEQ ID NO: 16
  MOD_RES (1)..(1): PYRROLIDONE CARBOXYLIC ACID

SEQ ID NO: 30 - protein, 40 aa, Homo sapiens; identical to SEQ ID NO: 17
  MOD_RES (1)..(1): PYRROLIDONE CARBOXYLIC ACID

SEQ ID NO: 31 - protein, 41 aa, Homo sapiens; identical to SEQ ID NO: 18
  MOD_RES (1)..(1): PYRROLIDONE CARBOXYLIC ACID

SEQ ID NO: 32 - protein, 28 aa, Homo sapiens
  MOD_RES (1)..(1): PYRROLIDONE CARBOXYLIC ACID
EVHHQKLVFFAEDVGSNKGAIIGLMVGG

SEQ ID NO: 33 - protein, 29 aa, Homo sapiens
  MOD_RES (1)..(1): PYRROLIDONE CARBOXYLIC ACID
EVHHQKLVFFAEDVGSNKGAIIGLMVGGV

SEQ ID NO: 34 - protein, 30 aa, Homo sapiens; identical to SEQ ID NO: 21
  MOD_RES (1)..(1): PYRROLIDONE CARBOXYLIC ACID

SEQ ID NO: 35 - protein, 31 aa, Homo sapiens; identical to SEQ ID NO: 22
  MOD_RES (1)..(1): PYRROLIDONE CARBOXYLIC ACID

SEQ ID NO: 36 - protein, 32 aa, Homo sapiens; identical to SEQ ID NO: 23
  MOD_RES (1)..(1): PYRROLIDONE CARBOXYLIC ACID

SEQ ID NO: 37 - protein, 33 aa, Homo sapiens; identical to SEQ ID NO: 24

SEQ ID NO: 38 - protein, 8 aa, Artificial sequence
  Epitope sequence
  MOD_RES (1)..(1): PYRROLIDONE CARBOXYLIC ACID
EFRHDSGC

SEQ ID NO: 39 - protein, 4 aa, Artificial sequence
  Epitope sequence
  MOD_RES (1)..(1): PYRROLIDONE CARBOXYLIC ACID
EVHH

SEQ ID NO: 40 - DNA, 436 bp, Mus musculus
atggtgtcctcagctcagttcctgtttctgttagtgctctggattcaggaaaccaacggtgatgttgtgatgacccagactccactcactttgtcggttaccattggacaaccagcctctatctcttgcaagtcaagtcagagcctcttatatagtgatggaaaaacctatttgaattggttattacagaggccaggccagtctccaatgcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccattcacgttcggctcggggacaaagttggaaataaaacgggctgatgctgcaccaactgtatccatcttcccaccat

SEQ ID NO: 41 - protein, 145 aa, Mus musculus
MVSSAQFLFLLVLWIQETNGDVVMTQTPLTLSVTIGQPASISCKSSQSLLYSDGKTYLNWLLQRPGQSPMRLIYLVSKLDSGVPDRFTGSGSGTDFTLKISRVEAEDLGVYYCVQGTHFPFTFGSGTKLEIKRADAAPTVSIFPP

SEQ ID NO: 42 - DNA, 440 bp, Mus musculus
atgggatggagcggggtctttctcttcctcctgtcaggaactgcaggtgtccactctgaggtccagctgcaacagtctggacctgagctggtgaagcctggagcttcaatgaagatatcctgcaaggcttctggttactcattcactggctataccatgaactgggtgaagcagagccatggaaagaaccttgagtggattggacttattaatccttacagtggtgttactaggtacaaccagaaattcaagggcaaggccacattaattgtagacaagtcatccagcacagcctacatggagctcctcagtctgacatctgaggactctgcagtctattattgtacaagagaggctaaacgggagtgggacgagacttactggggccaagggactctggtcactgtctctgcagccaaaacgacacccccatctgtcta

SEQ ID NO: 43 - protein, 146 aa, Mus musculus
MGWSGVFLFLLSGTAGVHSEVQLQQSGPELVKPGASMKISCKASGYSFTGYTMNWVKQSHGKNLEWIGLINPYSGVTRYNQKFKGKATLIVDKSSSTAYMELLSLTSEDSAVYYCTREAKREWDETYWGQGTLVTVSAAKTTPPSV

SEQ ID NO: 44 - DNA, 440 bp, Mus musculus
atggtgtccacagctcagttcctgtttctgttagtgctctggattcaggaaaccaacggtgatgttgtgatgacccagactccactcactttgtcggttaccattggacaaccagcctctatctcttgcaagtcaagtcagagcctcttatatagtgacggaaaaacctatttgaattggttattacagaggccaggccagtctccaatgcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccattcacgttcggctcggggacaaagttggaaataaaacgggctgatgctgcaccaactgtatccatcttcccaccatccag

SEQ ID NO: 45 - protein, 146 aa, Mus musculus
MVSTAQFLFLLVLWIQETNGDVVMTQTPLTLSVTIGQPASISCKSSQSLLYSDGKTYLNWLLQRPGQSPMRLIYLVSKLDSGVPDRFTGSGSGTDFTLKISRVEAEDLGVYYCVQGTHFPFTFGSGTKLEIKRADAAPTVSIFPPS

SEQ ID NO: 46 - DNA, 447 bp, Mus musculus
atgggatggagcggggtctttatcttcctcctgtcaggaactgcaggtgtccactctgaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtgttactaggtacaaccagaagttcaagggcaaggccacattaattgtagacaagtcatccagcacagcctacatggagctcctcagtctgacatctgaggactctgcagtctattactgtacaagagaggctaaacgggagtgggacgagacttactggggccaagggactctggtcactgtctctgcagccaaaacgacacccccatctgtctatccactg

SEQ ID NO: 47 - protein, 149 aa, Mus musculus
MGWSGVFIFLLSGTAGVHSEVQLQQSGPELVKPGASMKISCKASGYSFTGYTMNWVKQSHGKNLEWIGLINPYNGVTRYNQKFKGKATLIVDKSSSTAYMELLSLTSEDSAVYYCTREAKREWDETYWGQGTLVTVSAAKTTPPSVYPL

SEQ ID NO: 48 - DNA, 438 bp, Mus musculus
atgaagttgcctgttaggctgttggtgctggtgttctggattcctgtttccagcagtgatgttgtgatgacccagactccactctccctgcctgtcagtcttggagatcaagcctccatctcttgcagatctagtcagagccttgtacacagtgatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaacgggctgatgctgcaccaactgtatccatcttcccaccatccagt

SEQ ID NO: 49 - protein, 146 aa, Mus musculus
MKLPVRLLVLVFWIPVSSSDVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVPPTFGGGTKLEIKRADAAPTVSIFPPSS

SEQ ID NO: 50 - DNA, 456 bp, Mus musculus
atggactttgggctcagcttacttatttttgtccttattttaaaaggtgtccagtgtgaggtgaagctggtggagtctgggggaggcttagtgcagcctggagggtcccggaaactctcctgtgcagcctctggattcactttcagtgactacggaatggcgtgggttcgacaggctccagggaaggggcctgagtgggtagcattcattagtaatttggcatatagtatctactatgcagacactgtgacgggccgattcaccatctctagagagaatgccaagaacaccctgtacctggaaatgagcagtctgaggtctgaggacacagccatgtactactgtgcaaggtatgactacgataatatcttggactatgttatggactactggggtcaaggaacctcagtcaccgtctcctcagccaaaacaacacccccatcagtctatccactg

SEQ ID NO: 51 - protein, 152 aa, Mus musculus
MDFGLSLLIFVLILKGVQCEVKLVESGGGLVQPGGSRKLSCAASGFTFSDYGMAWVRQAPGKGPEWVAFISNLAYSIYYADTVTGRFTISRENAKNTLYLEMSSLRSEDTAMYYCARYDYDNILDYVMDYWGQGTSVTVSSAKTTPPSVYPL

SEQ ID NO: 52 - DNA, 432 bp, Mus musculus
atgaagttgcctgttaggctgttggtgctctggattcaggaaaccaagggtgatgttgtgctgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatgtggtgtctaaactggactctggagtccctgacaggttcactggcagtggatcaggaacagattttacactgaaaatcagcagagtggaggctgaggatttgggagtttattattgcgtgcaaggtacacattttccattcacgttcggctcggggacaaagttggaaataaaacgggctgatgctgcaccaactgtatccatcttcccaccatccagt

SEQ ID NO: 53 - protein, 144 aa, Mus musculus
MKLPVRLLVLWIQETKGDVVLTQTPLTLSVTIGQPASISCKSSQSLLYSNGKTYLNWLLQRPGQSPKRLIYVVSKLDSGVPDRFTGSGSGTDFTLKISRVEAEDLGVYYCVQGTHFPFTFGSGTKLEIKRADAAPTVSIFPPSS

SEQ ID NO: 54 - DNA, 435 bp, Mus musculus
atgggatggagcggggtctttctcttcctcctgtcagtaactgaaggtgtccactcccaggttcagctgcagcagtctggggctgagctggtgaggcctgggtcctcagtgaagatttcctgcaaggcttctggctatatattcaataactactggataaactgggtgaagcagaggcctggtcagggtcttgagtggattggacagatttatcctggagatggtgatactaactacaatgggaagttcaagggtaaagccacactgactgcagacaaatcctccagcacagcctacatgcagctcagcagcctaacatctgaggactctgcggtctatttctgtgcaagagagggatatattgtttattggggccaagggactctggtcactgtctctgcagccaaaacgacacccccatctgtctatccactg

SEQ ID NO: 55 - protein, 145 aa, Mus musculus
MGWSGVFLFLLSVTEGVHSQVQLQQSGAELVRPGSSVKISCKASGYIFNNYWINWVKQRPGQGLEWIGQIYPGDGDTNYNGKFKGKATLTADKSSSTAYMQLSSLTSEDSAVYFCAREGYIVYWGQGTLVTVSAAKTTPPSVYPL

SEQ ID NO: 56 - DNA, 441 bp, Mus musculus
atggagtcacatacccaggttcttatattgctgctgctatgggtatctggtacctgtggggacattgtgatgtcacagtctccatcctccctggctgtgtcagcaggagagaaggtcactatgagctgcaaatccagtcagagtctgttctacagtagaacccgaaagaactacttggcttggtaccaacagaaaccagggcagtctcctaaattgctgatctactgggcatccactagggaatctggggtccctgatcgcttcacaggcagtggatctgggacagatttcactctcaccatcagcagtgtgcaggctgaagacctggcagtttattactgcaagcaatcttacaatctgtggtcgttcggtggaggcaccaagctggaaatcaaacgggctgatgctgcaccaactgtatccatcttcccaccatccagt

SEQ ID NO: 57 - protein, 147 aa, Mus musculus
MESHTQVLILLLLWVSGTCGDIVMSQSPSSLAVSAGEKVTMSCKSSQSLFYSRTRKNYLAWYQQKPGQSPKLLIYWASTRESGVPDRFTGSGSGTDFTLTISSVQAEDLAVYYCKQSYNLWSFGGGTKLEIKRADAAPTVSIFPPSS

SEQ ID NO: 58 - DNA, 447 bp, Mus musculus
atgggatggagctgtatcatgttctttttggtagcaacagctacagatgtccactcccaggtccaactgcagcagcctgggactgaactggtgaagcctggggcttcagtgaagctgtcctgcaaggcttctggcttcaccttcaccagctactggatgcactgggtgagacagaggcctggacaaggccttgagtggattggagagattaatcctagtaacggtcgtactaactataatgagaagttcaagagcaaggccacactgactgtagacaaatcctccagcacagcctacatgcaactcagcagcctgacatctgaggactctgaggtctattactgtgcgagaggggatcttgcctgggactggtctgcttactggggccaagggactctggtcactgtctctgcagccaaaacgacacccccatctgtctatccactg

SEQ ID NO: 59 - protein, 149 aa, Mus musculus
  misc_feature (8)..(8): Xaa can be any naturally occurring amino acid
MGWSCIMXFLVATATDVHSQVQLQQPGTELVKPGASVKLSCKASGFTFTSYWMHWVRQRPGQGLEWIGEINPSNGRTNYNEKFKSKATLTVDKSSSTAYMQLSSLTSEDSEVYYCARGDLAWDWSAYWGQGTLVTVSAAKTTPPSVYPL

SEQ ID NO: 60 - DNA, 169 bp, Artificial sequence
  synthetic polynucleotide
agcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgcctt

SEQ ID NO: 61 - DNA, 255 bp, Artificial sequence
  synthetic polynucleotide
tatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaat

SEQ ID NO: 62 - DNA, 24 bp, Artificial sequence
  synthetic oligonucleotide
agcggataacaatttcacacagga

SEQ ID NO: 63 - DNA, 17 bp, Artificial sequence
  synthetic oligonucleotide
aaggcgattaagttggg

SEQ ID NO: 64 - DNA, 27 bp, Artificial sequence
  synthetic oligonucleotide
attgttgccgggaagctagagtaagta

SEQ ID NO: 65 - DNA, 24 bp, Artificial sequence
  synthetic oligonucleotide
tatgcagtgctgccataaccatga

SEQ ID NO: 66 - DNA, 24 bp, Artificial sequence
  synthetic oligonucleotide
tcctgtgtgaaattgttatccgct

SEQ ID NO: 67 - DNA, 24 bp, Artificial sequence
  synthetic oligonucleotide
agcggataacaatttcacacagga

(19) United States
(12) Patent Application Publication
(10) Pub. No.: US 2017/0363645 A1
(43) Pub. Date: Dec. 21, 2017

(54) Novel Method for the Detection of pGlu-Abeta Peptides

(71) Applicant: Probiodrug AG, Halle (Saale) (DE)

(72) Inventors: Martin Kleinschmidt, Halle (Saale) (DE); Torsten Hoffmann, Halle (Saale) (DE); Jens-Ulrich Rahfeld, Gemeinde Seegebiet Mansfelder Land (DE); Stephan Schilling, Halle (Saale) (DE); Beena Punnamoottil, Wiefelstede (DE); Michael Adler, Geestland (DE)

(21) Appl. No.: 15/536,014

(22) PCT Filed: Dec. 18, 2015
(86) PCT No.: PCT/EP2015/080518

§ 371 Date: Jun. 14, 2017

Related U.S. Application Data
(60) Provisional application No. 62/094,500, filed on Dec. 19, 2014.

(51) Int. Cl. G01N 33/68; C07K 16/18; C12Q 1/68

(52) U.S. Cl. CPC G01N 33/6896; C12Q 1/6804; C07K 16/18; G01N 2458/10; G01N 2333/4709; C07K 2317/14; C07K 2317/56; C07K 2317/34; G01N 2800/2821; G01N 2800/52

(57) ABSTRACT

The invention relates to a highly sensitive method for the detection of pGlu-Abeta (pGlu-Aβ) peptides and the use of this method in the diagnosis of neurodegenerative diseases, such as Alzheimer's disease and Mild Cognitive Impairment. The invention further concerns a novel method for monitoring the effectiveness of a treatment of neurode-generative diseases by monitoring changes in the level of pGlu-Aβ peptides.

### FIELD OF THE INVENTION

The invention relates to a highly sensitive method for the detection of pGlu-Abeta (pGlu-Aβ) peptides and the use of this method in the diagnosis of neurodegenerative diseases, such as Alzheimer's disease and Mild Cognitive Impairment. The invention further concerns a novel method for monitoring the effectiveness of a treatment of neurodegenerative diseases by monitoring changes in the level of pGlu-Aβ peptides.

### BACKGROUND OF THE INVENTION

Alzheimer's disease is the most common form of dementia and has a prevalence of approximately 65-70% among all dementia disorders (Blennow et al., 2006). Resulting from increased life expectancy, this disease has become a particular issue in highly developed industrialised countries like Japan and China as well as in the US and Europe. The number of Alzheimer patients is estimated to increase from 24 million in 2001 to 81 million in 2040 (Ferri et al., 2005). Currently, the costs for treatment and care of AD patients worldwide amount to approximately 250 billion US dollars per year.

The progression of the sporadic form of the disease is relatively slow and Alzheimer's disease will usually last for about 10-12 years after the onset of first symptoms. Presently, it is extremely difficult to make a reliable and early diagnosis of AD and distinguish it from other forms of dementia. A good diagnosis with a reliability of more than 90% is only possible in the later stages of the disease. Prior to that, it is only possible to make a prediction that Alzheimer's is possible or probable; diagnosis here relies on the use of certain criteria according to Knopman et al., 2001; Waldemar et al., 2007 or Dubois et al., 2007. Neurodegeneration starts however 20 to 30 years before the first clinical symptoms are noticed (Blennow et al., 2006; Jellinger K A, 2007). The onset of the clinical phase is usually characterized by the so-called “mild cognitive impairment” (MCI), where patients will show measurable cognitive deficits which are not sufficient to enable a diagnosis of a dementia disease in a clear fashion (Petersen et al., 1999; Chetkow et al., 2008). Many patients with MCI will have neuropathological changes which are typical for AD and which means that an earlier stage of AD is possible, but not certain (Scheff et al., 2006; Markesbery et al., 2006; Bouwman et al., 2007). There are however many MCI cases which will not progress to Alzheimer's; in these cases, other factors are responsible for the cognitive deficit (Saito et al., 2007; Jicha et al., 2006 and Petersen et al., 2006). While some MCI patients will not show any deterioration of their condition or even some kind of amelioration, for most MCI cases the cognitive deficit will continue to clinical dementia. The yearly rate of this conversion is approximately 10-19% (Gauthier et al., 2006; Fischer et al., 2007). At present there is a combination of clinical, neuropsychological and imaging processes which are capable of differentiating the various subtypes of Mild Cognitive Impairment (Devanand et al., 2007; Rossi et al., 2007; Whitwell et al., 2007; Panza et al., 2007). However, there is no significant difference between these subtypes in relation to the further progression of dementia (Fischer et al., 2007). Thus, it is of utmost importance to develop a method to enable a clear and reliable diagnosis of Alzheimer's disease in the early stages, suitably at its onset or during MCI.

### Prior Art Biomarkers

Biomarkers for Alzheimer's disease have already been described in the prior art. Alongside well known psychological tests such as e.g. ADAS-cog, MMSE, DemTect, SKT or the Clock Drawing test, biomarkers are supposed to improve diagnostic sensitivity and specificity for first diagnosis as well as for monitoring the progression of the disease. In relation to the current status of development of biomarkers for AD/MCI it was proposed to correlate the disease in the future with the other diagnostic criteria (Whitwell et al., 2007; Panza et al., 2007; Hyman S E, 2007). Biomarkers are supposed to support the classical neuro-psychological tests in the future. There is a common belief that they will be of great importance as surrogate markers for the development of agents against Alzheimer's (Blennow K, 2004; Blennow K, 2005; Hampel et al., 2006; Lewczuk et al., 2006; Irizarry M C, 2004).

### Structural Biomarkers

“Magnetic resonance imaging” (MRI) is an imaging process which allows detection of degenerative atrophies in the brain (Barnes J et al., 2007; Vemuri et al., 2008). Thus, atrophy of the medial temporal lobe (MTA) is sensitive to a degeneration of the hippocampal region in the brain of older patients; this can be made visible very clearly by MRI, but is not specific for Alzheimer's disease. Mild MTA is not encountered more frequently in other dementias (Barkhof et al., 2007) but it does correlate with MCI (Mevel et al., 2007). For this reason it is not possible to determine from MRI data alone whether the neurodegeneration is Alzheimer's disease or an early stage of Alzheimer's disease. A further imaging method is Positron Emission Tomography (PET) which visualises the accumulation of a detector molecule (PIB) on amyloid deposits. It could be detected that the thioflavin T-analogue (¹¹C)PIB will accumulate increasingly in certain regions of the brain of patients with MCI or mild Alzheimer's disease, respectively (Kemppainen et al., 2007; Klunk et al., 2004; Rowe et al., 2007); unfortunately this can also be detected in subjects who do not have dementia (Pike et al., 2007). This would probably indicate that the detection of amyloid deposits via PET allows detection of pre-clinical stages of Alzheimer's; however, this has to be confirmed by further studies. Besides the most frequently used processes, MRI and PET, there are additional structural biomarkers for AD: CBF-SPECT, CMRg1-PET (glucose metabolism proton spectroscopy (H-1 MRS), high field strength functional MRI, voxel-based morphometry, enhanced activation of the mediobasal temporal lobe (detected by fMRI, (R)-[(¹¹)C]PK11195 PET for the detection of microglial cells (Huang et al., 2007; Kantarci et al., 2007; Petrella et al., 2007; Hamalainen et al., 2007; Kircher et al., 2007; Kropholler et al., 2007).

### CSF Biomarkers

Senile plaques are one of the pathological characteristics of Alzheimer's disease. These plaques consist mostly of Aβ (1-42) peptides (Attems J, 2005). In some studies it could be shown that a low level of Aβ (1-42) in CSF of MCI patients correlates specifically with the further development of Alzheimer's disease in its progression (Blennow and Hampel, 2003; Hansson et al., 2006 and 2007). The reduction in CSF is probably due to enhanced aggregation of Aβ (1-42) in the brain (Fagan et al., 2006; Prince et al., 2004; Strozyk et al., 2003). Another possibility is the occurrence of semi-soluble Aβ (1-42) oligomers (Walsh et al., 2005) which would lead to a lower level of detection in CSF. In particular in the early stages of Alzheimer's, decreased concentrations of Aβ (1-42) would be detected, while increased amounts of Tau protein and phospho-tau proteins in CSF, respectively, could be detected (Ewers et al., 2007; Lewczuk et al., 2004). To provide a better predictability of biomarkers, it is usually attempted to use the Tau/Aβ (1-42) ratio and correlate it with the prediction of cognitive deficiency in older persons who do not have dementia (Fagan et al., 2007; Gustafson et al., 2007; Hansson et al., 2007; Li et al., 2007; Stomrud et al., 2007) as well as in MCI patients (Hampel et al., 2004; Maccioni et al., 2006; Schönknecht et al., 2007). A further correlation between ante mortem CSF level of Aβ (1-42), Tau, phospho-Tau-Thr231 and post-mortem histopathological alterations of the brain could be detected in AD patients (Clark et al., 2003; Buerger et al., 2006). In other studies, however, no correlation between CSF biomarkers and Aβ (1-42), total Tau and phospho-Tau with APOE ε4-allele, plaque and tangle load after autopsy could be detected (Engelborghs et al., 2007; Buerger et al., 2007). An interesting aspect was detected in a multicenter study. It appears that increased level of total Tau and phospho-Tau (181) correlates with a decreased ratio of Aβ (1-42)/Aβ (1-40), but not with the Aβ (1-42) alone (Wiltfang et al., 2007). An increased level of CSF Tau was however also detected in other CNS diseases such as Creutzfeldt-Jakob disease, brain infarction, and cerebral vascular dementia, which are all associated with a neuronal loss (Buerger et al., 2006 (2); Bibl et al., 2008). A further possible biomarker is the increase of BACE 1 activity in CSF as an indicator for MCI (Zhong et al., 2007). It is also discussed that the increased BACE 1 activity will result in increased Aβ production and therefore increased aggregation of the peptides. Alzheimer's disease is accompanied by neuroinflammatory processes. CSF anti-microglial cell antibodies are therefore possible biomarkers for these inflammatory processes in AD (McRea et al., 2007).

In spite of the multitude of biomarkers which are supposed to enable early diagnosis of Alzheimer's disease, there is not a single biomarker that ensures reliable and clear diagnosis.

This is usually because most studies use a comparison of the respective biomarkers and clinical diagnosis. A better approach would be the correlation of biomarkers with the pathological causes of Alzheimer's disease.

A possible approach would be repeated analysis of immuno-precipitated CSF samples of clearly identified and defined neuropathological dementia diseases to clarify whether Aβ (1-40) and Aβ (1-42) are in fact suitable neurochemical dementia markers (Jellinger et al., 2008). In order to discover novel, up to now unknown, biomarkers for Alzheimer's disease, CSF samples are usually analyzed via a comparative proteomic analysis which results in a diagnosis of AD with enhanced sensitivity and also to enable the differentiation from other degenerative dementia disorders (Finehout et al., 2007; Castano et al., 2006; Zhang et al., 2005; Simonsen et al., 2007; Lescuyer et al., 2004; Abdi et al., 2006). After a proteomic analysis, the potential new biomarker should be analyzed in detail for its suitability and correlation with pathological causes. A typical example for a biomarker which was found by a proteomic analysis is truncated cystatin C as a biomarker for multiple sclerosis; this biomarker was later proven to be a storage artefact (Irani et al., 2006; Hansson et al., 2007(2)).

### Plasma Biomarkers

Besides the frequently used plasma biomarkers, i.e. the Aβ peptides, further inflammatory plasma markers are used for the early diagnosis of dementia (Ravaglia et al., 2007; Engelhart et al., 2004) in particular for Alzheimer's (Motta et al., 2007). All of these are still under discussion. Further possible biomarkers were also found via comparative proteomic analysis of plasma from AD patients and healthy controls (German et al., 2007; Ray et al., 2007). The future will show whether these biomolecules are indeed specific for Alzheimer's disease and are suitable as biomarkers. There is no convincing or suitable data which would show either specificity or suitability of any of the biomarkers discussed above.

Contrary to the analysis of amyloid β in CSF, the results until now with respect to suitable Aβ biomarkers in plasma are not reliable or clear. In some studies a correlation between a decreased ratio of Aβ (1-42)/Aβ (1-40) in plasma and an enhanced conversion of cognitive normal persons to MCI or Alzheimer patients, respectively, was found ((Graff-Radford et al., 2007; van Oijen et al., 2006; Sundelof et al., 2008). Other studies however detected that a reduction of the Aβ (1-42) plasma level is more likely a marker for the conversion from MCI to AD (Song et al., 2007) and is not suitable as a marker for neurodegenerative purposes which are encountered with Alzheimer's (Pesaresi et al., 2006). Most of the studies however do not show a difference in Aβ plasma levels between healthy controls and patients with sporadic Alzheimer's (Fukumoto et al., 2003; Kosaka et al., 1997; Scheuner et al., 1996; Sobow et al., 2005; Tamaoka et al., 1996; Vanderstichele et al., 2000). Some studies also showed that the level of Aβ in plasma does not correlate with the level as encountered in the brain (Fagan et al., 2006; Freeman et al., 2007) nor does it correlate with the level encountered in CSF (Mehta et al., 2001; Vanderstichele et al., 2000). In a recent study, a correlation was detected for Aβ (1-40) and Aβ (1-42) between CSF and plasma, but only in healthy controls. This correlation could not be detected in MCI and AD which is explained by destroying the balance between CSF and plasma Aβ due to Aβ deposits in the brain (Giedraitis et al., 2007). Generally, it is assumed that plasma Aβ (1-42) level is not a reliable biomarker for MCI or AD (Blasko et al., 2008; Mehta et al., 2000; Brettschneider et al., 2005), whereas a decrease of the ratio plasma Aβ (1-38)/Aβ (1-40) is considered a biomarker for vascular dementia and comes close to the predictability of CSF markers (Bibl et al., 2007).

Moreover, Aβ oligomers are supposed to play a decisive role in initiating the neurodegenerative process (Walsh & Selkoe, 2007). In several studies, the neurotoxic effect was shown for Aβ dimers with 8 kDa to the point of protofibrils with over 100 kDa (Lambert et al., 1998; Walsh et al, 2002; Keayed et al., 2004; Cleary et al., 2005). Furthermore, such Aβ oligomers were found in human liquor (Pitschke et al., 1998; Santos et al., 2007; Klyubin et al., 2008). Besides their neurotoxicity, oligomers have also an influence on the determination of the Aβ concentration in human samples. The oligomerization leads to masking of the C-terminal epitopes of Aβ peptides (Roher et al., 2000) yielding to underestimated Aβ levels detected by C-terminal specific ELISA (Stenh et al., 2005). Englund et al., 2009, determined the Aβ 1-42 oligomer ratio in human CSF samples by measuring the Aβ 1-42 concentration under non-denaturing conditions via ELISA and under denaturing conditions using SDS-PAGE followed by Western Blot analysis. Another more common approach is the direct measurement of Aβ oligomers. Such a method, especially with oligomeric plasma Aβ as a biomarker, is however extremely difficult to establish as the Aβ peptides are very hydrophobic. Currently described assay systems use Aβ oligomer specific antibodies in a ELISA system (Englund et al., 2007; Schupf et al, 2008). However, the usage of ELISAs based on such oligomer specific antibodies have the same problems as traditional Aβ ELISA systems. The methods only achieve very unsatisfactory analytical sensitivity and encounter great problems with the very complex interactions between analytes and matrix, i.e. plasma. Usually, ELISA or ELISA-type systems (Multiplex) are used for quantification of Aβ, and recently also Aβ oligomers, in plasma. The specification of such detections systems is usually only unsatisfactorily analyzed or are completely disregarded. For example a critical item like the recovery rate is not analyzed or is not sufficiently investigated in the publications. The recovery rate is however decisive for giving a complete picture of those Aβ peptides or oligomers which occur in plasma. Differences between the studies can also result from the differences in these rates. A further important characteristic of an ELISA or multiplex system is its linearity. Thus, the concentrations determined for the analytes in plasma should only depend on the dilution used in the measurement to a very low degree or not at all. However, this is neither possible for ELISA nor for the multiplex systems for quantification of Aβ in plasma. Thus, the difference between the calculated plasma Aβ (1-42) concentration for a dilution of 1-20 was three times as high as for the 1-2 dilution of the same sample (Hansson et al., 2008). This example alone shows that the use of different dilutions of plasma samples in the several studies makes it impossible to compare the same.

Current methods used to diagnose AD involve analysis of cerebrospinal fluid (CSF) or brain tissue obtained from postmortem patients. Thus, among the markers currently under consideration are those related to the proteins, which account for the features found in Alzheimer brains postmortem. The neurofibrillary tangle is composed primarily of a hyperphosphorylated tau protein, a cytoskeletal protein. The neuritic plaque contains a core of amyloid protein, much of which is a 42-amino acid peptide (Aβ₄₂) derived from proteolytic cleavage of a larger precursor protein. Another form of this protein derived from the same precursor contains only 40 amino acids (Aβ₄₀). Deposits of this protein are found in the brains of AD victims. However, alterations in tau and the aforementioned beta amyloid peptides do not occur with sufficient frequency and magnitude so as to afford diagnostic value and therefore, blood tests based on these proteins do not seem to correlate well with AD. In addition to C-terminal variability, N-terminally modified Aβ peptides are abundant (Saido, T. C. et al. Dominant and differential deposition of distinct beta-amyloid peptide species, Aβ N3(pE), in senile plaques. Neuron 14, 457-466 (1995); Russo, C. et al. Presenilin-1 mutations in Alzheimer's disease. Nature 405, 531-532 (2000); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)). It appears that a major proportion of the Aβ peptides undergoes N-terminal truncation by two amino acids, exposing a glutamate residue, which is subsequently cyclized into pyroglutamate (pGlu or pE), resulting in pGlu-Aβ(3-42) peptides (Saido, T. C. et al. Dominant and differential deposition of distinct beta-amyloid peptide species, Aβ N3(pE), in senile plaques. Neuron 14, 457-466 (1995) ; Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)). Alternatively, pGlu may be formed following β′-cleavage by BACE1, resulting in pGlu-Aβ(11-42) (Naslund, J. et al. Relative abundance of Alzheimer Aβ amyloid peptide variants in Alzheimer disease and normal aging. Proc. Natl. Acad. Sci. U. S. A. 91, 8378-8382 (1994); Liu, K. et al. Characterization of Aβ (11-40/42) peptide deposition in Alzheimer's disease and young Down's syndrome brains: implication of N-terminally truncated Abeta species in the pathogenesis of Alzheimer's disease. Acta Neuropathol. 112, 163-174 (2006)). In particular pGlu-Aβ(3-42) has been shown to be a major constituent of Aβ deposits in sporadic and familial AD (Saido, T. C. et al. Dominant and differential deposition of distinct beta-amyloid peptide species, Aβ N3(pE), in senile plaques. Neuron 14, 457-466 (1995) ; Miravalle, L. et al. Amino-terminally truncated Aβ peptide species are the main component of cotton wool plaques. Biochemistry 44, 1081 0-1 0821 (2005)).

The pGluAβ(3-42)peptides coexist with Aβ(1-40/1-42) peptides (Saido, T. C. et al. Dominant and differential deposition of distinct beta-amyloid peptide species, Abeta N3pE, in senile plaques. Neuron 14, 457-466 (1995); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)), and, based on a number of observations, could play a prominent role in the pathogenesis of AD. For example, a particular neurotoxicity of pGluAβ(3-42) peptides has been outlined (Russo, C. et al. Pyroglutamate-modified amyloid beta-peptides—AbetaN3(pE)—strongly affect cultured neuron and astrocyte survival. J. Neurochem. 82, 1480-1489 (2002) and the pGlu-modification of N-truncated Aβ peptides confers resistance to degradation by most aminopeptidases as well as Aβ-degrading endopeptidases (Russo, C. et al. Pyroglutamate-modified amyloid beta-peptides—AbetaN3(pE)—strongly affect cultured neuron and astrocyte survival. J. Neurochem. 82, 1480-1489 (2002); Saido, T. C. Alzheimer's disease as proteolytic disorders: anabolism and catabolism of beta-amyloid. Neurobiol. Aging 19, S69-S75 (1998)). The cyclization of glutamic acid into pGlu leads to a loss of N-terminal charge resulting in accelerated aggregation of Aβ peptides having a pGlu residue at their N-terminus compared to the unmodified Aβ peptides (He, W. & Barrow, C. J. The Aβ 3-pyroglutamyl and 11-pyroglutamyl peptides found in senile plaque have greater beta-sheet forming and aggregation propensities in vitro than full-length Aβ. Biochemistry 38, 10871-10877 (1999); Schilling, S. et al. On the seeding and oligomerization of pGlu-amyloid peptides (in vitro). Biochemistry 45, 12393-12399 (2006)). Thus, reduction of pGlu-Aβ(3-42) formation should destabilize the peptides by making them more accessible to degradation and would, in turn, prevent the formation of higher molecular weight Aβ aggregates and enhance neuronal survival.

However, for a long time it was not known how the pGlu-modification of Aβ peptides occurs. The present Applicant discovered that glutaminyl cyclase (QC) is capable to catalyze pGlu-Aβ(3-42) formation under mildly acidic conditions, that specific QC inhibitors prevent pGlu-Aβ(3-42) generation in vitro and that, therefore, inhibition of glutaminyl cyclase is a novel therapeutic concept for the causative treatment of Alzheimer's disease (Schilling, S., Hoffmann, T., Manhart, S., Hoffmann, M. & Demuth, H.-U. Glutaminyl cyclases unfold glutamyl cyclase activity under mild acid conditions. FEBS Lett. 563, 191-196 (2004); Cynis, H. et al. Inhibition of glutaminyl cyclase alters pyroglutamate formation in mammalian cells. Biochim. Biophys. Acta 1764, 1618-1625 (2006); Schilling et al. Inhibition of glutaminyl cyclase—a novel therapeutic concept for the causative treatment of Alzheimer's disease. Nature Medicine 14, 1106-1111 (2008)).

The main problem associated with using pGlu-Aβ peptides as a biomarker for AD, MCI and NDS is that these peptides occur in high concentrations in senile plaques of the patients. I.e. the level of pGlu-Aβ peptides and/or changes in their level can only be determined by post mortem analysis of brain tissue. In contrast, only trace amounts or very low levels of pGlu-Aβ peptides can be found in other biological fluids, such as CSF, blood, plasma, serum or urine, which would allow a continuous monitoring of the pGlu-Aβ peptides during the life-time of the patients from time points prior to the onset of the diseases. However, present assay for pGlu-Aβ peptides are not sensitive enough for a robust detection and quantification these trace amounts. Accordingly, at present, there appears to be no satisfactory—diagnostic marker for manifested AD or MCI or for a subject, who, although exhibiting normal cognitive responses, inevitably, or most likely, is suspected to develop AD.

Age-Associated Cognitive Decline (AACD) and Mild Cognitive Impairment (MCI) are terms used to identify individuals who experience a cognitive decline that falls short of dementia.

These terms are equivalent, MCI being a more recently adopted term, and are used interchangeably throughout this application. Satisfaction of criteria (World Health Organization) for this diagnosis requires a report by the individual or family of a decline in cognitive function, which is gradual, and present at least 6 months. There may be difficulties across any cognitive domains (although memory is impaired in the vast majority of cases), and these must be supported by abnormal performance on quantitative cognitive assessments for which age and education norms are available for relatively healthy individuals (i.e., the patient is compared to normal subjects his/her own age). Performance must be at least 1 SD below the mean value for the appropriate population on such tests. Neither dementia, nor significant depression or drug effects may be present. No cerebral or systemic disease or condition known to cause cerebral cognitive dysfunction may be present. In Applicant's experience, all patients who were classified as CDR.5 (“questionable dementia”) on the Clinical Dementia rating scale and who met these exclusions, also met the criteria for AACD/MCI. About ⅓ of Alzheimer's patients have had a clearly definable period of isolated memory deficit which preceded their more global cognitive decline. (Haxby J. V., et al., Individual trajectories of cognitive decline in patients with dementia of the Alzheimer type, J. Clin. Exp. Neuropsychology 14:575-592, 1992.) Using AACD/MCI criteria, which look at other domains in addition to memory, the percentage with an identifiable prodrome is likely higher. Fortunately, not all AACD/MCI individuals seem to decline. It appears that a significant number of these subjects show a stable, non-progressive memory deficit on testing.

Attempts at predicting the onset of AD, MCI or NDS, or monitoring their progression have met with limited success. It has been discovered by the inventors of this application that an amount of a pGlu-Aβ peptide in a biological sample obtained from a subject that deviates from a reference amount in a control person can be positively correlated to a neurological disease state. Thus, the correlation of the presence of pGlu-Aβ peptide with the disease state represents a positive and more direct test for diagnosis in a patient suffering from one of the neurodegenerative diseases described above. The present invention is particularly based on the development of a novel assay method, which shows a dramatically improved sensitivity for the detection of pGlu-Aβ peptides in biological samples.

Accordingly, it is an objective of the present invention to provide an easily applicable biological sample test for predicting, diagnosing, or prognosticating AD and MCI using pGlu-Aβ peptides as a diagnostic marker. This easily applicable biological sample test is also suitable for monitoring the efficacy of novel treatments for AD and MCI.

Moreover, the present invention aims at providing pGlu-Aβ peptides as diagnostic markers which can be determined with reliable methods and can be used for reliable and clear prediction of AD and MCI.

### SUMMARY OF THE INVENTION

According to a first aspect of the invention there is provided a highly sensitive method for the detection of an Aβ target peptide in a biological sample, comprising a capture reagent which is specific for said Aβ target peptide; and an Aβ target peptide detection complex, said method comprising the steps of:
a) contacting a biological sample with said capture reagent and said detection complex; and
b) detecting said Aβ target peptide;

wherein the detection complex comprises an Aβ target peptide specific antibody and a nucleic acid marker.

Preferably, the Aβ target peptide is a pGlu-Aβ peptide.

According to a second aspect the invention provides the use of a the novel method for the detection of an Aβ target peptide, such as a pGlu-Aβ peptide in a biological sample in a method of diagnosing or monitoring a neurodegenerative disorder, such as Alzheimer's disease and Mild Cognitive Impairment.

According to a third aspect of the invention there is provided a method of diagnosing or monitoring a neurodegenerative disease, such as Alzheimer's disease and Mild Cognitive Impairment, which comprises determining the level of a pGlu-Aβ peptide in a biological sample from a test subject, comprising the following steps:
i. determining a first level of a pGlu-Aβ peptide in a biological sample from a subject suspected to be afflicted with said neurodegenerative disease with a method according to present invention;
ii. comparing the first level of the pGlu-Aβ peptide with a second level of said pGlu-Aβ peptide in a healthy control subject; and
iii. diagnosing the subject with a neurodegenerative disease where the level of said pGlu-Aβ peptide in said biological sample is increased compared to the level of said pGlu-Aβ peptide in the healthy control subject.

In a fourth aspect, the invention provides a method of monitoring the efficacy of a therapy in a subject having, suspected of having, or being predisposed to a neurodegenerative disease, such as Alzheimer's disease or Mild Cognitive Impairment, comprising determining determining the level of a pGlu-Aβ peptide in a biological sample from a test subject with a method according to present invention.

In a fifth aspect, the invention provides a kit for diagnosing a neurodegenerative disease, such as Alzheimer's disease or Mild Cognitive Impairment, which comprises at least one detection complex and instructions for using the kit, wherein said detection complex comprises a detection antibody capable of binding an Aβ peptide, one or more nucleic acid markers comprising a predetermined nucleotide sequence and one or more first linker molecules capable of specifically binding said antibody and the nucleic acid marker, and wherein at least one of the detection antibody and the capture antibody specifically binds to the pyroglutamate carrying amino terminus of a pGlu-Aβ peptide.

### Definitions

“Oligomeric” as used herein refers to a limited number of aggregated Aβ peptide monomer units. Examples of such oligomers include dimers, trimers and tetramers. The term “disaggregation” refers to the process of converting oligomeric forms of Aβ peptide to monomeric forms of Aβ peptide.

“Capture antibody” and “detection antibody” in the sense of the present application is intended to encompass those antibodies which bind to an Aβ peptide or a pGlu-Aβ peptide as the analyte.

Suitably the capture antibodies and detection antibodies bind to the Aβ peptide with a high affinity. In the context of the present invention, high affinity means an affinity with a K_{D} value of 10⁻⁷M or better, such as a K_{D} value of 10⁻⁸M or better or even more particularly, a K_{D} value of 10⁻⁹M to 10⁻¹²M.

The term “antibody” is used in the broadest sense and specifically covers intact monoclonal antibodies, polyclonal antibodies, multispecific antibodies (e.g. bispecific antibodies) formed from at least two intact antibodies, and antibody fragments as long as they exhibit the desired biological activity. The antibody may be an IgM, IgG (e.g. IgG1, IgG2, IgG3 or IgG4), IgD, IgA or IgE, for example. Suitably however, the antibody is not an IgM antibody. The “desired biological activity” is binding to a target Aβ peptide.

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single determinant on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Köhler et al., Nature, 256:495 (1975), or may be made by generally well known recombinant DNA methods. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain a minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences.

These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986), Reichmann et al, Nature. 332:323-329 (1988): and Presta, Curr. Op. Struct. Biel., 2:593-596 (1992). The humanized antibody includes a Primatized™ antibody wherein the antigen-binding region of the antibody is derived from an antibody produced by immunizing macaque monkeys with the antigen of interest or a “camelized” antibody.

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, N.Y., pp. 269-315 (1994).

The term “diabodies” refers to small antibody fragments with two antigen-binding sites, which fragments comprise a heavy-chain variable domain (V_{H}) connected to a light-chain variable domain (V_{D}) in the same polypeptide chain (V_{H}-V_{D}). By using a linker that is too short to allow pairing between the two domains on the same chain, the domains are forced to pair with the complementary domains of another chain and create two antigen-binding sites. Diabodies are described more fully in Hollinger et al., Proc. Natl. Acad. Sol. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In suitable embodiments, the antibody will be purified (1) to greater than 95% by weight of antibody as determined by the Lowry method, and most particularly more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, suitably, silver stain. Isolated antibody includes the antibody in situ within recombinant cells since at least one component of the antibody's natural environment will not be present. Ordinarily, however, isolated antibody will be prepared by at least one purification step.

As used herein, the expressions “cell”, “cell line,” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond. The terms “a”, “an” and “the” as used herein are defined to mean “one or more” and include the plural unless the context is inappropriate.

“Amyloid β, Aβ or β-amyloid” is an in the art recognized term and refers to amyloid β proteins and peptides, amyloid β precursor protein (APP), as well as modifications, fragments and any functional equivalents thereof. In particular, by amyloid β as used herein is meant any fragment produced by proteolytic cleavage of APP but especially those fragments which are involved in or associated with the amyloid pathologies including, but not limited to, Aβ (1-38) of SEQ ID NO: 1, Aβ (1-39) of SEQ ID NO: 2, Aβ (1-40) of SEQ ID NO: 3, Aβ (1-41) of SEQ ID NO: 4, Aα (1-42) of SEQ ID NO: 5, and Aβ (1-43) of SEQ ID NO: 6.

In the context of the present invention, “fragments of Aβ peptides” are all amyloid β peptides, which comprise a core amyloid β sequence of Aβ(11-38) of SEQ ID NO: 19. Further suitably for the purpose of the present invention are all amyloid β peptides, which comprise the core sequence of Aβ (15-38) of SEQ ID NO: 25. Such Aβ fragments, which comprise the amino acid sequence of Aβ (11-38) of SEQ ID NO: 19 or of Aβ (15-38) of SEQ ID NO: 25, have been shown to accumulate in a subject as a consequence of a neurodegenerative disorder, such as Alzheimer's disease and Mild Cognitive Impairment.

Further suitable examples for Aβ fragments are

| | |
|---|---|
| Aβ (2-38) | (SEQ ID NO: 7), |
| Aβ (2-39) | (SEQ ID NO: 8), |
| Aβ (2-40) | (SEQ ID NO: 9), |
| Aβ (2-41) | (SEQ ID NO: 10), |
| Aβ (2-42) | (SEQ ID NO: 11), |
| Aβ (2-43) | (SEQ ID NO: 12), |
| Aβ (3-38) | (SEQ ID NO: 13), |
| Aβ (3-39) | (SEQ ID NO: 14), |
| Aβ (3-40) | (SEQ ID NO: 15), |
| Aβ (3-41) | (SEQ ID NO: 16), |
| Aβ (3-42) | (SEQ ID NO: 17), |
| Aβ (3-43) | (SEQ ID NO: 18), |
| Aβ (11-38) | (SEQ ID NO: 19), |
| Aβ (11-39) | (SEQ ID NO: 20), |
| Aβ (11-40) | (SEQ ID NO: 21), |
| Aβ (11-41) | (SEQ ID NO: 22), |
| Aβ (11-42) | (SEQ ID NO: 23), and |
| Aβ (11-43) | (SEQ ID NO: 24. |

“Modified Amyloid β, Aβ or β-amyloid” encompasses all modifications at various amino acid positions in the amyloid β proteins and peptides, amyloid β precursor protein (APP), fragments and functional equivalents thereof. Useful in the present context are modifications at the N-and/or C-terminal amino acids of said amyloid β proteins and peptides, amyloid β precursor protein (APP), fragments and functional equivalents. Particularly useful are modifications at glutamine and glutamate residues, such as the cyclization of N-terminal glutamine or glutamate residues to pyroglutamate. Suitable examples according to the present invention are the amyloid β peptides of SEQ ID Nos. 13 to 24, which start with a glutamate residue at the N-terminus, wherein said the N-terminal glutamate residue is modified to pyroglutamate. Accordingly, particularly useful modified Aβ peptides are the “pGlu-Aβ peptides”.

“pGlu-Aβ peptides” in the context of the present invention relate to the following N-terminally pyroglutamated forms of Aβ and Aβ fragments:

| | |
|---|---|
| pGlu-Aβ (3-38) | (SEQ ID NO: 26), |
| pGlu-Aβ (3-39) | (SEQ ID NO: 27), |
| pGlu-Aβ (3-40) | (SEQ ID NO: 28), |
| pGlu-Aβ (3-41) | (SEQ ID NO: 29), |
| pGlu-Aβ (3-42) | (SEQ ID NO. 30), |
| pGlu-Aβ (3-43) | (SEQ ID NO: 31), |
| pGlu-Aβ (11-38) | (SEQ ID NO: 32), |
| pGlu-Aβ (11-39) | (SEQ ID NO: 33), |
| pGlu-Aβ (11-40) | (SEQ ID NO: 34), |
| pGlu-Aβ (11-41) | (SEQ ID NO: 35), |
| pGlu-Aβ (11-42) | (SEQ ID NO: 36), and |
| pGlu-Aβ (11-43) | (SEQ ID NO: 37). |

“Functional equivalents” encompass all those mutants or variants of Aβ peptides or pGlu-Aβ peptides which might naturally occur in the patient group which has been selected to undergo the method for detection or method for diagnosis as described according to the present invention. More particularly, “functional equivalent” in the present context means that the functional equivalents of Aβ peptides pGlu-Aβ peptides are mutants or variants thereof and have been shown to accumulate in Alzheimer's disease. The functional equivalents have no more than 30, such as 20, e.g. 10, particularly 5 and most particularly 2, or only 1 mutation(s) compared to the respective Aβ peptide or pGlu-Aβ peptide.

Particularly useful equivalents in the present context are those of Aβ (1-40) (SEQ ID NO. 2) and Aβ (1-42) (SEQ ID NO. 1), which are those described by Irie et al., 2005, namely the Tottori, Flemish, Dutch, Italian, Arctic and Iowa mutations of Aβ. Functional equivalents also encompass Aβ peptides derived from amyloid precursor protein bearing mutations next to the β- or γ-secretase cleavage site such as the Swedish, Austrian, French, German, Florida, London, Indiana and Australian variations (Irie et al., 2005).

The term “Aβ target peptide” includes “Amyloid β, Aβ or β-amyloid”, “fragments of Aβ peptides”, “Modified Amyloid β, Aβ or β-amyloid”, “pGlu-Aβ peptides” and “Functional equivalents” of the all of those. Preferably, the “Aβ target peptide” is a “pGlu-Aβ peptides”, fragment of functional equivalent thereof.

“Sandwich ELISAs” usually involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assay). For example, one suitable type of sandwich assay is an ELISA assay, in which case the detectable moiety is an enzyme.

The term “nucleic acid marker” or “nucleic acid reporter” refers to a nucleic acid molecule that will produce a detection product of a predicted size or other selected characteristic when used with appropriately designed oligonucleotide primers in a nucleic acid amplification reaction, such as a PCR reaction, preferably a real time PCR reaction. Skilled artisans will be familiar with the design of suitable oligonucleotide primers for PCR and programs are available, for example, over the Internet to facilitate this aspect of the invention (See, for example, http://bibiserv.techfak.uni-bielefeld.de/genefisher2/). A nucleic acid marker may be linear or circular. In specific embodiments, the nucleic acid marker will comprise a predetermined, linear nucleic acid sequence with binding sites for selected primers located at or near each end. In a circular DNA nucleic acid molecule, the primers will be internal rather than at an end, and a single primer may be used, e. g. for Rolling Circle Amplification. Amplified DNA may be detected using any available method, including, but not limited to techniques such as labeled oligonucleotide probes, SYBR Green or ethidium bromide staining or electrochemical methods. In certain embodiments, the DNA sequence located between the primer binding sites comprises a “characteristic identification sequence” capable of being detected during the PCR reaction. Fluorescent signal generation may, for example, be sequence-specific (Molecular Beacons, Taq Man, Scorpions, fluorogenic primers, such as the LUX primers (Invitrogen (Carlsbad, Calif.)) or mass dependent (SYBR Green, Ethidium Bromide). The examples provided are not meant to be an exhaustive list of possible nucleic acid detection schemes as those skilled in the art will be aware of alternative markers suitable for use in the methods of the present invention.

The term “characteristic identification sequence” refers to a nucleic acid sequence that can be specifically detected by virtue of hybridization to oligonucleotide or other nucleic acid that has been labeled with a detectable marker such as a radioisotope, a dye (such as a fluorescent dye), or other species that will be known in the art. In some embodiments, the characteristic identification sequence is capable of binding a “molecular beacon” probe. The term “molecular beacon” refers to oligonucleotides such as those sold by Operon Technologies (Alameda, Calif., USA) and Synthetic Genetics (San Diego, Calif., USA). (See also, Tyagi and Kramer (1996), Nat. Biotechnol, 14: 303-308; and Tyagi et al. (2000), Nat Biotechnol, 18:1191-96). In another specific embodiment, the identification sequence is capable of binding a Scorpion. “Scarpions” are bifunctional molecules containing a PCR primer covalently linked to a probe. The fluorophore in the probe interacts with a quencher which reduces fluorescence. During a PCR reaction the fluorophore and quencher are separated which leads to an increase in light output from the reaction tube. Scorpions are sold by DxS Ltd. (Manchester, UK). As noted herein, a signal can be generated using a variety of techniques and reagents.

The terms “polynucleotide” and “nucleic acid (molecule)” are used interchangeably to refer to polymeric forms of nucleotides of any length, including naturally occurring and non-naturally occurring nucleic acids. The polynucleotides may contain deoxyribonucleotides, ribonucleotides and/or their analogs. Methods for selection and preparation of nucleic acids are diverse and well described in standard biomolecular protocols. A typical way would be preparative PCR and chromatographic purification starting from existing template DNAs or stepwise synthesis of artificial nucleic acids.

Nucleotides may have any three-dimensional structure, and may perform any function, known or unknown. The term “nucleic acid molecule” includes single-, double-stranded and triple helical molecules. “Oligonucleotide” refers to polynucleotides of between 3 and about 100, for example 3-50, 5-30, or 5-20 nucleotides of single- or double-stranded nucleic acid, typically DNA.

Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis.

The following are non-limiting embodiments of nucleic acids: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1- methylpseudouracil, 1-methylguanine, 1-methylinosine, 2,2-dimethylguanine, 2-methyladenine, 2-methylguanine,3-methylcytosine,5-methylcytosine, pseudouracil, 5-pentylnyluracil and 2,6-diaminopurine. The use of uracil as a substitute for thymine in a deoxyribonucleic acid is also considered an analogous form of pyrimidine. A nucleic acid may also include a backbone modification, wherein the phosphodiester bonds are replaced with phosphorothioates or methylphosphonates.

The term “linker” or “linker molecule” refers to a molecule that either links the nucleic acid marker to the non-nucleic acid receptor and thus facilitates detection of an analyte specifically bound by the non-nucleic acid receptor via detecting the nucleic acid marker or that interconnects other linker molecules. The linker molecules according to the present invention are chemically distinct from the non-nucleic acid receptor and the nucleic acid marker and are capable of binding the non-nucleic acid receptor and the nucleic acid marker and/or other, chemically different linker molecules. To achieve formation of a detection complex according to the invention, the linker molecules of the invention are at least bivalent, preferably trivalent, tetravalent, pentavalent, hexavalent or multivalent. In this connection, the term “multivalent” relates to linker molecules that can bind more than 2, preferably more than 3 other molecules. The multiple molecules bound by the linker molecules may be the same or different. For example, a linker molecule may have binding sites for the nucleic acid marker, the non-nucleic acid receptor and/or another, chemically different linker molecule or, alternatively, 2, 3, 4 or more binding sites for one specific binding partner. In the latter case, complex formation is achieved by coupling one or more binding partner(s) to other components of the detection complex, such as the nucleic acid marker, the non-nucleic acid receptor and another, chemically different linker molecule. In this connection, the expression “binding partner” relates to a molecule which is specifically recognized and bound by a linker molecule. The binding partner may thus be a small organic molecule, but can also be any other molecule, such as, for example, a peptide, polypeptide, protein, saccharide, polysaccharide or a lipid or an antigen or hapten. Specific examples for such a pair of linker molecule and binding partner are the streptavidin/biotin and avidin/biotin binding pairs. If the linker molecule is streptavidin/avidin and the binding partner is biotin, the biotin may be coupled to either one or all of the non-nucleic acid receptor, the nucleic acid marker and the second linker molecule to facilitate detection complex formation. The binding of the linker molecule to its binding partner and/or the nucleic acid marker, the non-nucleic acid receptor and/or other, chemically distinct linker molecules is preferably non-covalent. The linker molecules according to the invention may comprise one or more molecules selected from the group consisting of polysaccharides, organic polymers, polypeptides and nucleic acids distinct from the nucleic acid marker. In case the linker molecule according to the invention comprises a nucleic acid distinct from the nucleic acid marker, the linker molecule may further comprise a polysaccharide, organic polymer or polypeptide chemically coupled to the nucleic acid part.

The term “organic polymers”, as used herein, refers to polymers of organic molecules, preferably including functional groups such as hydroxy, amino, imino, nitro, cyano, carboxy, carbonyl, carbamid, halo, acylhalo, aldehyde, epoxy, and/or thiol groups. Exemplary polymers are, for example, polyethyleneimines, poly(meth)acrylamides, polyamines, polyamidoamines, polyethyleneglycols, polyethylene, polypropylene, poly(meth)acrylates, polyurethanes, polystyrenes, and polyesters. Preferred are cationic polymers, such as those having amino or imino groups, such as, for example, polyethyleneimines, poly(meth)acrylamides, polyamines, and polyamidoamines. The organic polymers may be linear, branched or dendritic.

The term “polysaccharide” refers to molecules consisting of at least two monosaccharides linked by a glycosidic bond and includes disaccharides and oligosaccharides. Exemplary polysaccharides are starch, glycogen, dextran, cellulose and chitin. The polysaccharides according to the invention may be linear, branched or dendritic polysaccharides.

The terms “contacting” or “incubating” as used interchangeably herein refer generally to providing access of one component, reagent, analyte or sample to another. For example, contacting can involve mixing a solution comprising a non-nucleic acid receptor with a sample. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples. In one embodiment of the invention, contacting involves adding a solution comprising a non-nucleic acid receptor to a sample utilizing a delivery apparatus, such as a pipette-based device or syringe-based device.

The term “detecting” as used herein refers to any method of verifying the presence of a given molecule. The techniques used to accomplish this may include, but are not limited to, PCR, sequencing, PCR sequencing, molecular beacon technology, Scorpions technology, hybridization, and hybridization followed by PCR. Examples of reagents which might be used for detection include, but are not limited to, radiolabeled and fluorescently oligonucleotide probes and dyes, such as DNA intercalating dyes.

The term “detection” as used herein refers to two or more molecules, which have been linked together. The linkage to each other may be covalent or non-covalent. One example of a detection according to the invention is a detection consisting of a non-nucleic acid receptor and a nucleic acid marker, non-covalently linked to each other by means of a first linker molecule. In a particular embodiment, the detection comprises, consists essentially of or consists of a biotinylated DNA molecule coupled via a streptavidin molecule to an analyte-specific biotinylated antibody. Such a detection may be an oligomeric detection, i.e. comprise more than one nucleic acid marker and/or more than one non-nucleic acid receptor and/or more than one first linker molecules.

The term “detection complex”, as used herein, refers to a complex of one or more non-nucleic acid receptors, one or more nucleic acid markers, one or more linker molecules of a first type, and one or more linker molecules of a second type. In one embodiment, the detection complexes according to the invention may comprise two or more detections as defined above and additionally one or more second linker molecule(s). In one specific embodiment of the invention, such a detection complex according to the invention comprises at least two non-nucleic acid receptors and at least two nucleic acid markers, non-covalently linked to each other by means of at least two first and at least two second linker molecules. In a particular embodiment, the detection comprises, consists essentially of or consists of at least one, for example 2 or more, biotinylated DNA molecule(s) coupled via at least one, preferably two or more, streptavidin molecule(s) and at least one, preferably to or more, biotinylated organic polymer or protein molecules, such as BSA, polyethyleneimines, poly(meth)acrylamides, polyamines, or polyamidoamines, to at least one, preferably two or more, analyte-specific biotinylated antibody/antibodies. In a particular embodiment, the detection complex comprises, consists essentially of or consists of one or more, preferably at least two (bis-)biotinylated DNA marker molecule(s) coupled via one or more, preferably at least two streptavidin molecule(s) and one or more, preferably at least two poly-biotinylated organic polymer(s) or protein(s)/polypeptide(s), such as BSA, polyethyleneimines, poly(meth)acrylamides, polyamines, or polyamidoamines, to one or more, preferably at least two analyte-specific poly-biotinylated antibodies. In this connection, “poly-biotinylated” refers to covalent modification with two or more biotin moieties.

### BRIEF DESCRIPTION OF THE FIGURES

FIG. 1: shows the results of the investigation of 3 pan-specific β-amyloid antibodies (6E10, 13-28, 12F4) with different capture antibodies (clones 6-1-6, 17-4-3, 24-2-3 (all three Probiodrug AG) and clone 2-48 (Synaptic Systems).
1 Detection antibody 6E10, positive control of 300 pg/ml pGlu-Aβ(3-42) peptide (SEQ ID NO: 30),
2 Detection antibody 6E10, negative control
3 Detection antibody 13-28 (BAM 90.1, Sigma), positive control of 300 pg/ml pGlu-Aβ(3-42) peptide (SEQ ID NO: 30),
4 Detection antibody 13-28, negative control
5 Detection antibody 12F4 (Covance), positive control of 300 pg/ml pGlu-Aβ(3-42) peptide (SEQ ID NO: 30),
6 Detection antibody 12F4, negative control

FIG. 2: shows the recovery plot for high and low concentrations of standard pGlu-Aβ(3-42) peptide (SEQ ID NO: 30) mixtures used in the validation of the pGlu-Aβ(3-42) peptide (SEQ ID NO: 30).

FIG. 3: DemTect Test

Mean values (Mean±SD) of the results of classification differences in AD patients and healthy subjects (Group I: 18-30 years; Group II: 31-45 years; Group III: 46-65 years) by DemTect Scale.

FIG. 4: Mini-Mental-State Test

Mean values (Mean±SD) of the results of classification differences in AD patients and healthy subjects (Group I: 18-30 years; Group II: 31-45 years; Group III: 46-65 years) by Mini-Mental-State Test.

FIG. 5: Clock-Drawing Test

Mean values (Mean±SD) of the results of classification differences in AD patients and healthy subjects (Group I: 18-30 years; Group II: 31-45 years; Group III: 46-65 years) by Clock-Drawing Test.

### DETAILED DESCRIPTION OF THE INVENTION

According to a first aspect of the invention there is provided a highly sensitive method for the detection of an Aβ target peptide in a biological sample, comprising a capture reagent which is specific for said Aβ target peptide; and an Aβ target peptide detection complex, said method comprising the steps of:
a) contacting a biological sample with said capture reagent and said detection complex; and
b) detecting said Aβ target peptide;
   wherein the detection complex comprises an Aβ target peptide specific antibody and a nucleic acid marker.

Preferably, said Aβ target peptide is a pGlu-Aβ peptide.

Suitably, said detection complex consists of, consists essentially of or comprises a detection antibody capable of binding a pGlu-Aβ peptide, one or more nucleic acid markers comprising a predetermined nucleotide sequence; and one or more first linker molecules adapted to bind said antibody and the nucleic acid marker.

In a preferred embodiment, the method according to the present invention comprises the steps of:
a) contacting the biological sample with the detection complex under conditions allowing the binding of an Aβ target peptide to said detection complex; and
b) subsequently contacting said Aβ target peptide, which is bound to the detection complex, with a capture reagent capable of binding an Aβ target peptide under conditions allowing the binding of said capture reagent to said Aβ target peptide.

Suitably, the capture reagent is a capture antibody specific for a pGlu-Aβ peptide.

According to a further preferred aspect of the invention there is provided a method for the detection of a pGlu-Aβ peptide in a biological sample, comprising the steps of:
a) contacting a biological sample with at least one detection complex, wherein said detection complex consists of, consists essentially of or comprises a detection antibody capable of binding an Aβ target peptide, one or more nucleic acid markers comprising a predetermined nucleotide sequence and one or more first linker molecules adapted to bind said antibody and the nucleic acid marker; under conditions allowing the binding of said detection complex to said Aβ target peptide;
b) further contacting said Aβ target peptide, which is bound to the detection complex, with a capture antibody capable of binding said Aβ target peptide under conditions allowing the binding of said capture antibody to said Aβ target peptide; and
c) detecting said Aβ target peptide;
   wherein said Aβ target peptide is a pGlu-Aβ peptide; and wherein at least one of the detection antibody and the capture antibody specifically binds to the pyroglutamate carrying amino terminus of said pGlu- Aβ peptide.

The data presented herein surprisingly demonstrate that the sensitivity of the detection of pGlu-Aβ peptides in biological samples was significantly increased by the method of invention, i.e. trace amounts down to 4.2 fg/ml of could be detected with high reliability. The limit of quantitation (LOQ) for the detection of pGlu- Aβ peptides could be improved at least 1000 fold compared to existing assay methods in the prior art.

The biological samples concerned by the present invention usually comprise a mixture of different Aβ peptides, fragments or functional derivatives thereof as well as different pGlu- Aβ peptides, fragments or functional derivatives thereof. For example, the biological samples may comprise a mixture of the peptides according to SEQ ID NOs: 1 to 37.

In one embodiment of the method invention, both of the detection antibody and the capture antibody specifically bind to the pyroglutamate carrying amino terminus of said pGlu-Aβ peptide. The advantage of this embodiment is that already in the step of (i) contacting a biological sample with at least one detection complex, wherein said detection complex comprises a detection antibody, only pGlu-Aβ peptides, e.g. the pGlu-Aβ peptides of at least one of SEQ ID NOs: 26-37 are bound by the detection antibody. As a result, there is already made a selective enrichment of said pGlu-Aβ peptides in the first step of the method of the invention. This embodiment of the method of the invention is particularly suitable for the detection of pGlu-Aβ peptides, which are comprised in Aβ oligomers.

In an alternative embodiment of the method of the invention, the capture antibody specifically binds to the pyroglutamate carrying amino terminus of said pGlu-Aβ peptide and the detection antibody binds to another epitope sequence of an Aβ peptide. The advantage of this embodiment is that in the step (i) of contacting a biological sample with at least one detection complex, wherein said detection complex comprises a detection antibody, all Aβ peptides, pGlu-Aβ peptides as well as fragments and functional variants thereof, which present in said biological sample, are bound by the detection antibody and are thus enriched in this method step. The highly selective discrimination between Aβ peptides and pGlu-Aβ peptides is then performed in method step ii) of further contacting the Aβ peptide, which is bound to the detection complex, with a capture antibody capable of binding a pGlu-Aβ peptide under conditions allowing the binding of said capture antibody to said pGlu-Aβ peptide. This alternative embodiment is especially advantageous when the pGlu-Aβ peptides are comprised not only in Aβ oligomers, but when the pGlu-Aβ peptides are comprised in the biological samples as free monomers or as monomers bound to proteins contained in the biological samples. This alternative embodiment is particularly advantageous when only one pGlu-Aβ monomer is bound to a protein contained in the biological samples. When both, the detection antibody and the capture antibody specifically bind to the same epitope such as to the pyroglutamate carrying amino terminus of said pGlu-Aβ peptide, monomeric pGlu-Aβ peptide bound to the could possibly not detected by the capture antibody, because the pyroglutamate carrying amino terminus of said pGlu-Aβ peptide is already masked or occupied by the detection antibody in the detection complex.

In a further alternative embodiment of the method of the invention, the detection antibody specifically binds to the pyroglutamate carrying amino terminus of said pGlu-Aβ peptide and the capture antibody binds to another epitope sequence of an Aβ peptide. This embodiment is as advantageous as the afore described embodiment for the detection of free or protein-bound monomeric pGlu-Aβ peptide and pGlu-Aβ containing oligomers.

The “other epitope sequence” of an Aβ peptide, to which the capture antibody and/or the detection antibody binds, when the capture antibody and/or the detection antibody do not bind to the pyroglutamate carrying amino terminus of a pGlu-Aβ peptide, may be a part of the amino acid sequence of a full-length Aβ peptide, such as Aβ (1-38) of SEQ ID NO: 1, Aβ (1-39) of SEQ ID NO: 2, Aβ (1-40) of SEQ ID NO: 3, Aβ (1-41) of SEQ ID NO: 4, Aβ (1-42) of SEQ ID NO: 5, and Aβ (1-43) of SEQ ID NO: 6. The capture antibody and/or the detection antibody, which do not bind to the pyroglutamate carrying amino terminus, may specifically detect the untruncated and/or unmodified N-terminus or C-terminus of an Aβ peptide. Further suitably, the other epitope sequence may be part of the amino acid sequence of one of SEQ ID NOs: 7 to 37.

In a preferred embodiment of the method of the invention, the other epitope sequence of an Aβ peptide, to which the capture antibody and/or the detection antibody binds, when the capture antibody and/or the detection antibody do not bind to the pyroglutamate carrying amino terminus of a pGlu-Aβ peptide, is comprised in the core amyloid β sequence of Aβ (11-38) of

SEQ ID NO: 19 in the case that pGlu-Aβ peptides starting with the N-terminal pGlu residue at position 3, most preferably the pGlu-Aβ peptides of SEQ ID NOs: 26-31 shall be detected and/or quantified.

In a preferred embodiment of the method of the invention, the other epitope sequence of an Aβ peptide, to which the capture antibody and/or the detection antibody binds, when the capture antibody and/or the detection antibody do not bind to the pyroglutamate carrying amino terminus of a pGlu-Aβ peptide, is comprised in the core amyloid β sequence of Aβ(15-38) of SEQ ID NO: 25 in the case that pGlu-Aβ peptides starting with the N-terminal pGlu residue at position 11, most preferably the pGlu-Aβ peptides of SEQ ID NOs: 32-37 shall be detected and/or quantified.

Suitably, the other epitope sequence consists of the entire amino acid sequence of an Aβ peptide of one of SEQ ID NOs: 1 to 25. More suitably, other epitope sequence consists of 30, 25, 20 or 15 amino acids of an Aβ peptide of one of SEQ ID NOs: 1 to 25. Most preferably, the other epitope sequence consists of 10, 9, 8, 7, 6, 5, 4 or 3 amino acids of an Aβ peptide of one of SEQ ID NOs: 1 to 25.

Particularly good and reliable results are achieved with the method of the present invention, when the detection complex is provided in a matrix similar to the biological sample. Further suitably, the detection complex comprised in such a matrix similar to the biological sample is added directly to the biological sample. Surprisingly, best results can be obtained when the detection complex is provided in a matrix similar to the biological sample, is added directly to the biological in a ratio <1+1.

The method of the present invention is based on a new and surprising strategy in the assay protocol, which comprises a combined overnight incubation of the biological sample and the addition of the detection complex, which is contained in a matrix similar to the biological sample, directly to the biological sample in a ratio of 1+0.03. This assay protocol is quite unexpected and unconventional compared to methods used in the prior art, where a typical sample dilution is 1+1 to 1+9 and higher. Only this incubation strategy enabled the intended highly sensitive detection of the Aβ target peptide.

A further increase in the sensitivity and reliability of the method of the present invention is achieved, when a mixture of different pGlu- Aβ peptides is applied to human CSF or artificial human CSF as a reference substance for quantification. In a preferred embodiment, a 1+1 mixture of pGlu-Aβ(x-40) and pGlu-Aβ(x-42) is applied to human CSF or artificial human CSF as a reference substance for quantification, wherein x is an integer selected from 3 and 11. Most preferably, a 1+1 mixture of pGlu-Aβ(3-40) and pGlu-Aβ(3-42) is applied human CSF or artificial human CSF as a reference substance for quantification, when the Aβ target peptide is selected from SEQ ID NOs: 1 to 25. Yet most preferably, a 1+1 mixture of pGlu-Aβ(11-40) and pGlu-Aβ(11-42) is applied human CSF or artificial human CSF as a reference substance for quantification, when the Aβ target peptide is selected from SEQ ID NOs: 32-37. Such a use of a mixture of two Aβ target peptides as a reference standard is a new and innovative strategy.

Suitable examples for detection and/or capture antibodies, which do not bind to the pyroglutamate carrying amino terminus of pGlu-Aβ peptides are:
3D6, Epitope:1-5 (Elan Pharmaceuticals, Innogenetics),
pAb-EL16, Epitope: 1-7,
2H4, Epitope: 1-8 (Covance),
1E11, Epitope: 1-8 (Covance),
20.1, Epitope: 1-10 (Covance, Santa Cruz Biotechnology),
Rabbit Anti-Aβ Polyclonal Antibody, Epitope: 1-14 (Abcam),
AB10, Epitope: 1-16 (Chemicon/Upstate—part of Millipore),
82E1, Epitope: 1-16 (IBL),
pAb 1-42, Epitope: 1-11,
NAB228, Epitope: 1-11 (Covance, Sigma-Aldrich, Cell Signaling, Santa Cruz Biotechnology, Zymed/Invitrogen),
DE2, Epitope: 1-16 (Chemicon/Upstate—part of Millipore),
DE2B4, Epitope: 1-17 (Novus Biologicals, Abcam, Accurate, AbD Serotec),
6E10, Epitope: 1-17 (Signet Covance, Sigma-Aldrich),
10D5, Epitope: 3-7 (Elan Pharmaceuticals),
WO-2, Epitope: 4-10 (The Genetics Company),
1A3, Epitope 5-9 (Abbiotec),
pAb-EL21, Epitope 5-11,
310-03, Epitope 5-16 (Abcam, Santa Cruz Biotechnology),
Chicken Anti-Human Aβ Polyclonal Antibody, Epitope 12-28 (Abcam),
Chicken Anti-Human Aβ Polyclonal Antibody, Epitope 25-35 (Abcam),
Rabbit Anti-Human Aβ Polyclonal Antibody, Epitope: N-terminal (ABR),
Rabbit Anti-Human Aβ Polyclonal Antibody (Anaspec),
12C3, Epitope 10-16 (Abbiotec, Santa Cruz Biotechnology),
16C9, Epitope 10-16 (Abbiotec, Santa Cruz Biotechnology),
19B8, Epitope 9-10 (Abbiotec, Santa Cruz Biotechnology),
pAb-EL26, Epitope: 11-26,
BAM90.1, Epitope: 13-28 (Sigma-Aldrich),
Rabbit Anti-beta-Amyloid (pan) Polyclonal Antibody, Epitope: 15-30 (MBL),
22D12, Epitope: 18-21 (Santa Cruz Biotechnology),
266, Epitope: 16-24 (Elan Pharmaceuticals),
pAb-EL17; Epitope: 15-24,
4G8, Epitope: 17-24 (Covance),
Rabbit Anti-Aβ Polyclonal Antibody, Epitope: 22-35 (Abcam),
G2-10; Epitope: 31-40 (The Genetics Company),
Rabbit Anti-Aβ, aa 32-40 Polyclonal Antibody (GenScript Corporation),
EP1876Y, Epitope: x-40 (Novus Biologicals),
G2-11, Epitope: 33-42 (The Genetics Company),
16C11, Epitope: 33-42 (Santa Cruz Biotechnology),
21F12, Epitope: 34-42 (Elan Pharmaceuticals, Innogenetics),
1A10, Epitope: 35-40 (IBL),
D-17 Goat anti-Aβ antibody, Epitope: C-terminal (Santa Cruz Biotechnology),
2C8, Epitope: 1-16 (Accurate),
BAM-10, Epitope: 1-12 (Biotrend, Sigma-Aldrich),
12B2, Epitope: 11-28 (IBL, Biotrend),
6F/3D, Epitope: 8-17 (Accurate),
310-01, Epitope: 10-16, (Accurate),
11A5-10, Epitop: 34-40, (Millipore),
12F4, Epitope: 36-42, (Millipore, Covance),
9C4, Epitope: 37-43, (Milipore),
7N22, Epitope: 1-20, (Biosource),
11A50-B10, Epitope: 35-40) (Covance),
G2-13, Epitope: C-terminus Aβ42 (Genetics Company),
2B9, Epitope: 1-17 (Santa Cruz), and
9C4, Epitope: 3-8 (Covance).

The pGlu-Aβ peptide, which is preferably detected by the method of the invention, is at least one pGlu-Aβ peptide selected from the group consisting of SEQ ID NOs: 26 to 37.

In a preferred embodiment of the invention, said detection antibody and/or said capture antibody is a monoclonal antibody, more preferably a humanized monoclonal antibody. Further preferred according to the invention is a detection antibody and/or a capture antibody, which is a diabody or a single chain antibody which retains the high affinity.

According to one embodiment of the invention, the capture and/or the detection antibody, which specifically binds to the pyroglutamate carrying amino terminus of said pGlu-Aβ peptides of SEQ ID NOs: 26-31, is selected from the group consisting of
pGlu3-Aβ antibody clone 2-48 (monoclonal, mouse); Synaptic Systems,
pGlu3-Aβ antibody (polyclonal, rabbit); Synaptic Systems, Biotrend, IBL,
pGlu3-Aβ antibody clone 8E1 (monoclonal, mouse); Anawa,
pGlu3-Aβ antibody clone 8E1 (monoclonal, mouse); Biotrend,
Anti-Human Amyloidlβ (N3pE) Rabbit IgG (polyclonal, rabbit); IBL,
Abeta-pE3 rabbit polyclonal, affinity purified, Synaptic systems,
Anti-Human Aβ N3pE (8E1) Mouse IgG Fab (monoclonal, mouse); IBL,
pGlu3-Aβ antibody clone 337.48 (monoclonal, mouse); Biolegend,
pGlu3-Aβ antibody clone 1-57 (monoclonal, mouse); Synaptic Systems,
pGlu3-Aβ antibody clone 70D7 (monoclonal, mouse); Synaptic Systems, and
oligo pGlu3-Aβ antibody clone 9D5 (monoclonal, mouse); Synaptic Systems.

In a further preferred embodiment of the invention, the capture and/or the detection antibody, specifically binds to the epitope sequence pGlu-FRHDSGC, SEQ ID NO: 38.

In a more preferred embodiment of the invention, the detection and/or the capture antibody is produced by a hybridoma cell line selected from the group consisting of:

| | |
|---|---|
| Aβ 5-5-6 | (Deposit No. DSM ACC 2923), |
| Aβ 6-1-6 | (Deposit No. DSM ACC 2924), |
| Aβ 17-4-3 | (Deposit No. DSM ACC 2925), and |
| Aβ 24-2-3 | (Deposit No. DSM ACC 2926), |

which are disclosed in WO 2010/009987.

In a most preferred embodiment of the invention, the variable part of the light chain of said detection antibody and/or said capture antibody has the nucleotide sequence of SEQ ID NO: 40 or the amino acid sequence of SEQ ID NO: 41, and the variable part of the heavy chain of said detection antibody and/or said capture antibody has the nucleotide sequence of SEQ ID NO: 42, or the amino acid sequence of SEQ ID NO: 43.

In a further most preferred embodiment of the invention, the variable part of the light chain of said detection antibody and/or said capture antibody has the nucleotide sequence of SEQ ID

NO: 44 or the amino acid sequence of SEQ ID NO: 45, and the variable part of the heavy chain of said detection antibody and/or said capture antibody has the nucleotide sequence of SEQ ID NO: 46, or the amino acid sequence of SEQ ID NO: 47.

In a further most preferred embodiment of the invention, the variable part of the light chain of said detection antibody and/or said capture antibody has the nucleotide sequence of SEQ ID NO: 48 or the amino acid sequence of SEQ ID NO: 49, and the variable part of the heavy chain of said detection antibody and/or said capture antibody has the nucleotide sequence of SEQ ID NO: 50, or the amino acid sequence of SEQ ID NO: 51.

In a further most preferred embodiment of the invention, the variable part of the light chain of said detection antibody and/or said capture antibody has the nucleotide sequence of SEQ ID NO: 52 or the amino acid sequence of SEQ ID NO: 53, and the variable part of the heavy chain of said detection antibody and/or said capture antibody has the nucleotide sequence of SEQ ID NO: 54, or the amino acid sequence of SEQ ID NO: 55.

In a preferred embodiment of the invention, the capture and/or the detection antibody, which specifically binds to the pyroglutamate carrying amino terminus of said pGlu-Aβ peptides of SEQ ID NOs: 32 to 37, is selected from the group consisting of
pGlu11-Aβ antibody clone 173D8, (monoclonal, mouse); Synaptic Systems, and
pGlu11-Aβ antibody (polyclonal rabbit); Synaptic Systems.

In a further preferred embodiment of the invention, the capture and/or the detection antibody, specifically binds to the epitope sequence pGlu-VHH, SEQ ID NO: 39.

More preferably, said detection antibody and/or said capture antibody is a monoclonal antibody produced by hybridoma cell line Aβ (Deposit No. DSM ACC 3100), which is disclosed in WO 2012/123562.

In a most preferred embodiment of the invention, the variable part of the light chain of said detection antibody and/or said capture antibody has the nucleotide sequence of SEQ ID NO: 56 or the amino acid sequence of SEQ ID NO: 57, and the variable part of the heavy chain of said detection antibody and/or said capture antibody has the nucleotide sequence of SEQ ID NO: 58, or the amino acid sequence of SEQ ID NO: 59.

It is further preferred according to invention, that the capture antibody is immobilized on a solid support, and thereafter the detection complex comprising the detection antibody and the analyte binds to the capture antibody, thus forming an insoluble complex.

General methods for preparing a detection complex, which is used in the method of the invention, has been described in DE 199 41 756 A1 and EP 2 189 539 A1, the disclosure of which is incorporated herein in their entirety. In particular, it has been found that by use of detection complexes that consist of, consist essentially of or comprise one or more non-nucleic acid receptors (e.g. an antibody such as a detection antibody), one or more nucleic acid markers, one or more first linker molecules adapted to bind the non-nucleic acid receptor and the nucleic acid marker, and one or more second linker molecules adapted to bind the first linker molecule the performance, in particular the assay sensitivity and the signal-to-background-ratio, of an Immuno-PCR (IPCR) reaction can be significantly improved.

According to a further aspect of the invention there is provided a method for the detection of a pGlu-Aβ peptide in a biological sample, comprising the steps of:
i. contacting a biological sample with at least one detection complex, wherein said detection complex consist of, consist essentially of or comprises a detection antibody capable of binding an Aβ peptide, one or more nucleic acid markers comprising a predetermined nucleotide sequence and one or more first linker molecules adapted to bind said antibody and the nucleic acid marker; and one or more second linker molecules adapted to bind the first linker molecule under conditions allowing the binding of said detection complex to the Aβ peptide;
ii. further contacting the Aβ peptide, which is bound to the detection complex, with a capture antibody capable of binding an Aβ peptide under conditions allowing the binding of said capture antibody to said Aβ peptide; and
iii. detecting said pGlu-Aβ peptide;
   wherein at least one of the detection antibody and the capture antibody specifically binds to the pyroglutamate carrying amino terminus of said pGlu-Aβ peptide.

Thus, in one embodiment, the invention relates to detection complexes comprising one or more detection antibody molecules, one or more nucleic acid markers, one or more first linker molecules adapted to bind the non-nucleic acid receptor and the nucleic acid marker, and one or more second linker molecules adapted to bind the first linker molecule. In one specific embodiment of the present invention, the detection complexes comprise a plurality of detection antibody molecules, nucleic acid markers, first linker molecules and second linker molecules. It is desirable to include several detection antibodies with specific binding affinity for a certain Aβ peptide or pGlu-Aβ peptide in the detection complexes according to the invention in order to enhance the affinity for the analyte of choice by means of increased avidity. In turn, it is also desirable to include several nucleic acid markers in the detection complexes, because thus the positive signal, indicating the presence of the analyte in a sample, is enhanced and the signal-to-background ratio improved.

In the detection complexes according to the present invention, the first and second linker molecules serve the purpose to form supramolecular aggregates of the detection antibodies and the nucleic acid markers and thus increase the sensitivity of the complexes as detection reagents in IPCR assays. To achieve the self-assembly of supramolecular networks, the first linker molecules are adapted to bind the detection antibodies, the nucleic acid markers and the second linker molecules.

The supramolecular detection complexes according to the present invention may include 2-50, preferably 5-50 molecules of each the detection antibodies, the nucleic acid markers, the first linker molecules and the second linker molecules. In one embodiment of the invented detection complexes, the complexes include at least 2, preferably 3 or more detection antibody molecules and/or nucleic acid markers. In some embodiments of the invention, the invented detection complexes include about 10-40 nucleic acid marker and first linker molecules, about 5-15 detection antibody molecules and about 5-10 second linker molecules.

In accordance with a further embodiment of the present invention, the detection antibody may be an antibody fragment or functional variant of a detection antibody or detection antibody fragment that retains the ability to specifically bind an Aβ peptide or pGlu-Aβ peptide. The detection antibody may be a monoclonal or polyclonal antibody and the antibody fragment may be, for example, a Fab or F(ab′)2 fragment, a single chain variable fragment (scFv), an Fv diabody or a linear antibody. The detection antibodies, fragments or functional variants thereof may be biotinylated and thus include one or more biotin or biotin analog moieties.

The nucleic acid marker including a predetermined nucleotide sequence may be any nucleic acid, such as, for example, double- or single-stranded DNA, double- or single stranded RNA, or double-stranded hybrids of DNA and RNA. The nucleic acid marker may contain nucleotide analogs, such as those, in which the naturally occurring bases and sugars are replaced by base analogs or sugar analogs or in which the phosphate backbone is substituted by other suitable groups. Suitable modifications have been mentioned above. All afore-mentioned nucleic acid marker molecules may be biotinylated and thus include one or more biotin or biotin analog moieties. One particular example are mono- or bis-biotinylated DNA molecules.

In one embodiment of the invention, the detection complexes are formed by non-covalent interactions between the first linker molecules and the detection antibody and/or the nucleic acid marker. In such an embodiment, the binding of the first linker molecule to the second linker molecule may also be non-covalent.

According to one specific embodiment of the present invention, the binding of the first linker molecule to the detection antibody, the nucleic acid marker and/or the second linker molecule may be facilitated by coupling each the non-nucleic acid receptor, the nucleic acid marker and/or the second linker molecule to one or more, for example 2, 3, 4, 5 or more binding partners of the first linker molecule. These binding partners may be the same or different for the detection antibody, the nucleic acid marker and the second linker molecule. In one embodiment of the invention, these binding partners of the first linker molecule are covalently coupled to the detection antibody, the nucleic acid marker and/or the second linker molecule.

In accordance with one specific embodiment of the present invention, the binding partner of the first linker molecule may be a ligand of the first linker molecule. It is preferred that the first linker molecule is bivalent, trivalent, tetravalent or multivalent for the binding to the binding partner. In one embodiment, the first linker molecule specifically recognizes and binds its binding partner with a high affinity.

In one embodiment of the present invention, the first linker molecule may be avidin or streptavidin or a biotin-binding fragment or mutant thereof.

In a specific embodiment, the binding partner of the first linker molecule is biotin or a biotin analog. The biotin analogs of the present invention preferably retain the ability to specifically bind to avidin, streptavidin or a biotin-binding fragment or mutant thereof.

If the first linker molecule is avidin, streptavidin or a biotin-binding fragment or mutant thereof, the binding of the first linker molecule to the detection antibody, the nucleic acid marker and/or the second linker molecule may be facilitated by coupling the detection antibody, the nucleic acid marker and/or the second linker molecule to biotin or a biotin analog. This coupling may be covalent and either of the detection antibody, the nucleic acid marker and/or the second linker may be coupled to at least 2 biotin or biotin analog molecules.

In an alternative embodiment, the first linker molecule may be a fusion protein or an at least bivalent antibody or antibody-like molecule adapted to simultaneously bind at least two of the detection antibodies, the nucleic acid marker and the second linker molecule.

According to one embodiment of the invention, the second linker molecules may be selected from the group consisting of nucleic acids distinct from the nucleic acid marker, organic polymers, polypeptides and polysaccharides. In one embodiment of the present invention, the second linker molecules comprise at least two, three or four different molecules selected from the group consisting of nucleic acids distinct from the nucleic acid marker, organic polymers, proteins and polysaccharides.

If the second linker molecules consist of, consist essentially of or include organic polymer molecules, these may be selected from the group consisting of cationic polymers, such as linear, branched or dendritic polyethyleneimines, polyacrylamides, polyamines, and polyamidoamines according to one specific embodiment of the present invention.

In case the second linker molecules consist of, consist essentially of or include protein or polypeptide molecules, these may be selected from the group consisting of serum albumines and immunoglobulins or fragments thereof. In one embodiment, the second linker molecule may be BSA. Alternatively, the second linker molecules may be homo-polymers of cationic amino acids, such as poly-lysine, poly-histidine or poly-arginine.

In one alternative embodiment of the present invention, the second linker molecules consist of, consist essentially of or include polysaccharides selected from the group consisting of linear, cyclic or branched dextrans.

In still another embodiment of the present invention, the second linker molecules may also consist of, consist essentially of or include nucleic acid molecules distinct from the nucleic acid marker. The nucleic acid molecules may be nucleic acid oligomers, for example, oligonucleotides or nucleic acid polymers, such as polynucleotides. Exemplary nucleic acid oligomers that may be used as second linker molecules consist of two complementary nucleic acid strands, wherein each of these strands is independently adapted to bind to a first linker molecule. In one specific embodiment of the invention, this binding to a first linker molecule is facilitated by covalently coupling each single strand of the nucleic acid oligomer to one first linker molecule, with the result that each of these two strands is independently coupled to a first linker molecule by a covalent bond.

Another alternative embodiment may be a polynucleotide adapted to bind one or more first linker molecules.

In one embodiment of the present invention, the second linker molecules may also be a heterogeneous mixture of the above specified molecules. According to one embodiment of the present invention, the second linker molecules thus include two or more different molecules selected from the group consisting of linear, branched or dendritic polyethyleneimines, polyacrylamides, polyamines, polyamidoamines, homo-polymers of cationic amino acids, such as poly-lysine, serum albumines, immunoglobulins or fragments thereof, linear, cyclic or branched dextrans, poly- and oligonucleotides. In one specific embodiment of the present invention, the second linker molecules include nucleic acid oligomers consisting of two complementary nucleic acid strands, wherein each of these strands is independently adapted to bind to a first linker molecule, optionally be forming a covalent bond, and organic polymers, such as polyethyleneimines, polypeptides, such as albumines or immunoglobulins, polysaccharides and/or polynucleotides distinct from the nucleic acid oligomers and the nucleic acid marker.

All afore-mentioned second linker molecules may be coupled to one or more biotin or biotin analog molecules. Specific examples of second linker molecules according to the invention are polybiotinylated BSA, polybiotinylated polyethyleneimine, polybiotinylated poly(meth)acrylamide, polybiotinylated polyamine, or polybiotinylated polyamidoamine.

In one embodiment of the present invention, the detection complexes of the invention may further include one or more modulators adapted to bind to the first linker molecules. These modulators are used to saturate non-occupied binding sites of the first linker molecule for the detection antibody, the nucleic acid marker, the second linker molecule and/or a binding partner of the first linker molecule. In order to avoid that the modulators compete with the binding of the detection antibody, the nucleic acid marker, the second linker molecule and/or a binding partner of the first linker molecule coupled to the detection antibody, the nucleic acid marker and/or the second linker molecule, the modulator is preferably added after formation of a detection complex from the detection antibody, the nucleic acid marker, the first and the second linker molecule. The modulators may be positively charged and may be selected from the group consisting of amino-biotin, diamino-biotin and amino-substituted biotin analogs.

In a further aspect, the present invention relates to methods for the preparation of the above detection complexes. In one embodiment, such a method for the preparation of a detection complex according to the invention includes the steps of:
(a) contacting one or more nucleic acid markers with one or more first linker molecules adapted to bind nucleic acid markers and detection antibodies to form a complex of one or more nucleic acid markers with one or more first linker molecules;
(b) contacting the complex of step (a) with one or more detection antibodies to form a complex of one or more detection antibodies, one or more nucleic acid markers and one or more first linker molecules; and
(c) contacting the complex of step (b) with one or more second linker molecules adapted to bind the first linker molecules to form a complex of one or more detection antibodies, one or more nucleic acid markers, one or more first linker molecules and one or more second linker molecules.

This method may optionally further include the step of:
(d) contacting the complex of step (c) with one or more modulators adapted to bind to the first linker molecules to saturate non-occupied binding sites of the first linker molecule for the detection antibody, the nucleic acid marker and the second linker molecule to form a complex of one or more detection antibodies, one or more nucleic acid markers, one or more first linker molecules, one or more second linker molecules and one or more modulators.

In another embodiment, the invention encompasses a method for the preparation of a detection complex including:
(i) one or more detection antibodies capable of specifically binding an Aβ peptide and/or a pGlu-Aβ peptide;
(ii) one or more nucleic acid markers including a predetermined nucleotide sequence;
(iii) one or more first linker molecules adapted to bind the detection antibody and the nucleic acid marker;
(iv) one or more nucleic acid oligomers adapted to bind the first linker molecules, wherein the one or more nucleic acid oligomers comprise two complementary nucleic acid strands distinct from the nucleic acid marker; and
(v) one or more organic polymers, polynucleotides distinct from the nucleic acid marker and the one or more nucleic acid oligomers, polypeptides or polysaccharides adapted to bind the first linker molecules;
   wherein the method comprises the steps of:
(a) contacting one nucleic acid strand of the one or more nucleic acid oligomers with one or more first linker molecules to form a first detection of one or more first linker molecules and one nucleic acid strand of the one or more nucleic acid oligomers;
(b) contacting the nucleic acid strand of the one or more nucleic acid oligomers complementary to that used in step (a) with one or more first linker molecules to form a second detection of one or more first linker molecules and one nucleic acid strand of the one or more nucleic acid oligomers complementary to that used in step (a);
(c) contacting the detection of step (a) with one or more nucleic acid markers to form a first complex of one or more first linker molecules detectiond to one nucleic acid strand of the one or more nucleic acid oligomers and one or more nucleic acid markers;
(d) contacting the detection of step (b) with one or more detection antibodies to form a second complex of one or more first linker molecules detectiond to one nucleic acid strand of the one or more nucleic acid oligomers complementary to that used in step (a) and (c) and one or more detection antibodies;
(e) contacting the first complex of step (c) with one or more organic polymers, polynucleotides, polypeptides or polysaccharides, to form a third complex of one or more first linker molecules detectiond to one nucleic acid strand of the one or more nucleic acid oligomers, one or more nucleic acid markers and one or more organic polymers, polynucleotides, polypeptides or polysaccharides; and
(f) contacting the second complex of step (d) with the third complex of step (e) to form a complex detection of one or more first linker molecules detectiond to one nucleic acid strand of the one or more nucleic acid oligomers, one or more nucleic acid markers, one or more organic polymers, polynucleotides, polypeptides or polysaccharides, one or more first linker molecules detectiond to one nucleic acid strand of the one or more nucleic acid oligomers complementary to that used in step (a) and (c) and one or more detection antibodies.

In one embodiment of the invention, this method may further include the step of contacting the complexes of steps (d) and (e) with one or more modulators adapted to bind to the first linker molecule before step (f).

In the methods of the invention, the detection antibody, the nucleic acid marker, the first linker molecule, the second linker molecule and the modulator may be as defined above. In particular, the binding of the detection antibody, the nucleic acid marker and the second linker molecule to the first linker molecule may be facilitated by one or more binding partner(s) of the first linker molecule coupled to the detection antibody, the nucleic acid marker and the second linker molecule. In one embodiment, these binding partners are biotin and/or a biotin analog and the first linker molecule is streptavidin, avidin or a biotin-binding fragment thereof.

Also encompassed by the present invention are the detection complexes obtainable by the invented methods.

In another aspect, the invention is also directed to the use of the detection complex according to the invention in an immunoassay for the detection or the determination of the amount of a pGlu-Aβ peptide. Said pGlu-Aβ peptide may be as defined above and is specifically recognized and bound by the detection antibody. The immunoassay may include a nucleic acid amplification reaction to amplify the nucleic acid marker. The amplification reaction is preferably a polymerase chain reaction (PCR), more preferably a real-time PCR reaction.

In still another aspect, the invention features a method for detecting a pGlu-Aβ peptide in a sample, wherein the method includes the steps of:
(a) contacting a detection complex according to the invention comprising one or more detection antibodies capable of specifically binding said pGlu-Aβ peptide with said sample to form a complex of said analyte and said detection complex;
(b) specifically detecting the presence of the one or more nucleic acid markers in said complex; wherein the presence of the one or more nucleic acid markers indicates the presence of the pGlu-Aβ peptide in said sample.

In one embodiment of the present invention, the detecting step (b) may comprise amplifying the one or more nucleic acid markers in a PCR reaction, preferably a real time PCR reaction.

In one embodiment, the detection of the pGlu-Aβ peptide includes the determination of the amount of the pGlu-Aβ peptide, that is a quantitative determination of the pGlu-Aβ peptide.

Detection and, in a specific embodiment, also quantitation of the pGlu-Aβ peptide may be achieved by detection and, optionally, quantitation of the number of amplicons generated in the PCR reaction using the nucleic acid marker as a template. Detection and, optionally quantitation may be achieved by using nucleic acid probes labeled with a detectable label or suitable dyes.

In one embodiment of the invention, the nucleic acid marker is detected by real time PCR, carried out in a commercially available instrument. Real-time PCR amplification is performed in the presence of a fluorescent-labelled probe which specifically binds to the amplified PCR product, for example a dual labelled primer including a fluorescent moiety quenched by another label which is in spatial proximity to the fluorescent label as long as the primer is not incorporated in an amplification product and separated from each other due to elongation of the primer during amplification.

In another embodiment, a non-primer detectable probe which specifically binds the PCR amplification product is used. The probe may include a covalently bonded reporter dye at the 5′-end and a downstream quencher dye at the 3′-end, which allows fluorescent resonance energy transfer (FRET).

Detection of the amplified PCR product may be carried out after each amplification cycle, as the amount of PCR product is at every stage of the amplification reaction proportional to the initial number of template copies. The number of template copies can be calculated by means of the detected fluorescence of the reporter dye. In an intact probe the fluorescence is quenched due to the close proximity of the reporter dye and quencher dye. During PCR, the nuclease activity of the DNA polymerase cleaves the probe in the 5′-3′ direction and thus separates the reporter dye from the quencher dye. Because reporter and quencher dye are then no longer in close proximity to each other, the fluorescence of the reporter dye is increased. The increase in fluorescence is measured and is directly proportional to the amplification during PCR. See Heid et al. (1996), “Real time quantitative PCR” Genome Research 6(10):986-994. This detection system is now commercially available as the TaqMan® PCR system from Perkin-Elmer, which allows real time PCR detection.

In an alternative embodiment, the reporter dye and quencher dye may be located on two separate probes which hybridize to the amplified PCR detector molecule in adjacent locations sufficiently close to allow the quencher dye to quench the fluorescence signal of the reporter dye (Rasmussen et al. (1998), “Quantitative PCR by continuous fluorescence monitoring of a double strand DNA specific binding dye” Biochemica 2:8-15). As with the detection system described above, the 5′-3′ nuclease activity of the polymerase cleaves the one dye from me probe containing it, separating the reporter dye from the quencher dye located on the adjacent probe preventing quenching of the reporter dye. As in the embodiment described above, detection of the PCR product is by measurement of the increase in fluorescence of the reporter dye.

In other embodiments of this invention, other real time PCR detection strategies may be used, including known techniques such as intercalating dyes (ethidium bromide) and other double stranded DNA binding dyes used for detection (e.g. SYBR green, FMC Bioproducts), dual fluorescent probes (Wittwer et al. (1977) BioTechniques 22:130-138 and Wittwer et al. (1997) BioTechniques 22:176-181) and panhandle fluorescent probes (i.e. molecular beacons; Tyagi and Kramer (1996) Nature Biotechnology 14:303-308). Although intercalating dyes and double stranded DNA binding dyes permit quantitation of PCR product accumulation in real time applications, they suffer from a lack of specificity, detecting primer dimer and any non-specific amplification product. Careful sample preparation and handling, as well as careful primer design, using known techniques are necessary to minimize the presence of matrix and contaminant DNA and to prevent primer dimer formation. Appropriate PCR instrument analysis software and melting temperature analysis permit a means to extract specificity (Ririe, K., et al. (1977) Anal. Biochem. 245:154-160) and may be used with these embodiments.

In still another embodiment of this invention, the Scorpions reaction is used as a real time PCR detection method. Scorpions are bi-functional molecules containing a PCR primer covalently linked to a probe. The fluorophore in the probe interacts with a quencher which reduces fluorescence. During the PCR reaction the primer binds to the template and is elongated by the polymerase. Once the elongation reaction is completed and primer and template are separated in the denaturation step, the elongated primer sequence can interact intramolecularly with the probe sequence in the next annealing step. The binding of the probe to the elongated primer sequence prevents interaction of the probe-bound fluorophore with the quencher, which leads to an increase in light output from the reaction tube. Currently, there are two formats for Scorpions, the bimolecular Scorpion format and the unimolecular format. In the bimolecular format the quencher is bound to a separate nucleic acid molecule which is complementary to the probe sequence, whereas in the unimolecular format both, fluorophore and quencher, are attached to the same molecule, and an integral stem loop sequence is used to bring the quencher close to the fluorophore.

The Scorpions technique is described more fully in Whitcombe et al. (1999), Detection of PCR products using self-probing amplicons and fluorescence, Nature Biotech 17, pages 804-807. This detection system is now commercially available as the scorpions system from DxS Ltd. (Manchester, UK).

The design of primers for the amplification reaction and nucleic acid probes is well-established in the art and thus routine practice for the skilled person. Suitable fluorescent reporter dyes are also known and commercially available, and include, without limitation 6-carboxy-fluorescein (FAM), tetrachloro-6-carboxy-fluorescein (TET), 2,7-dimethoxy-4,5-dichloro-6-carboxy-fluorescein (JOE) and hexachloro-6-carboxy-fluorescein (HEX). Another suitable reporter dye is 6-carboxy-tetramethylrhodamine (TAMRA).

In another aspect, the invention relates to a kit i.e., a packaged combination of reagents in predetermined amounts with instructions for performing the detection method or the diagnostic method of the invention. Such a kit comprises one or more detection complexes according to the invention or manufactured according to the methods of the invention. Such a kit may additionally contain further components. Exemplary components that may be additionally comprised in the kits of the present invention include, but are not limited to stabilizers, buffers (e.g. a block buffer or lysis buffer), dyes, oligonucleotide primers or probes, which may be optionally labelled with a detectable label, etc. The components of the detection complexes according to the invention may be as defined above.

Furthermore, the antibodies used in the methods of the present invention can also be provided in the kit.

The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

According to a further aspect of the invention, there is provided a kit for diagnosing a neurodegenerative disorder, such as Alzheimer's disease which comprises detection complexes according to the invention or manufactured according to the methods of the invention, at least one capture antibody and instructions for use. In one embodiment, the kit additionally comprises at least one capture antibody that specifically binds to the pyroglutamate carrying amino terminus of said pGlu-Aβ peptide.

In still another aspect, the invention is also directed to the use of one or more organic polymer, polypeptide, polysaccharide and/or oligo- or polynucleotide molecules, all of which may be optionally biotinylated, as additional linker molecules in a detection comprising one or more non-nucleic acid receptors, one or more nucleic acid markers and one or more first linker molecules to form a detection complex comprising one or more non-nucleic acid receptors, one or more nucleic acid markers, one or more first linker molecules and one or more organic polymer, polypeptide, polysaccharide and/or oligo- or polynucleotide molecules.

The biological sample may be any sample, for example from a human. In one specific example, the sample is a tissue sample, a body fluid sample or a cell sample. In one embodiment, the biological sample is selected from the group consisting of blood, serum, urine, cerebrospinal fluid (CSF), plasma, lymph, saliva, sweat, pleural fluid, synovial fluid, tear fluid, bile and pancreas secretion. In a further embodiment, the biological sample is plasma. In a preferred embodiment, the biological sample is CSF.

The biological sample can be obtained from a patient in a manner well-known to a person skilled in the art. In particular, a blood sample can be obtained from a subject and the blood sample can be separated into serum and plasma by conventional methods. The subject, from which the biological sample is obtained is preferably a subject suspected of being afflicted with Alzheimer's disease, at risk of developing Alzheimer's disease and/or being at risk of or having any other kind of dementia. In particular, the sample is obtained from a subject suspected of having Mild Cognitive Impairment (MCI) and/or being in the early stages of Alzheimer's disease.

The invention further relates to the use of the method for the detection of a pGlu-Aβ peptide according to the present invention in a method of diagnosing or monitoring a neurodegenerative disease, such as Alzheimer's disease and Mild Cognitive Impairment.

In particular, the invention provides a method of diagnosing or monitoring a neurodegenerative disease, such as Alzheimer's disease and Mild Cognitive Impairment, which comprises determining the level of a pGlu-Aβ peptide in a biological sample from a subject, comprising the following steps:
i. determining a first level of a pGlu-Aβ peptide in a biological sample from a subject suspected to be afflicted with said neurodegenerative disease with a method for the detection of a pGlu-Aβ peptide according to the present invention;
ii. comparing the first level of the pGlu-A Aβ peptide with a second level of said pGlu-Aβ peptide in a healthy control subject; and
iii. diagnosing the subject with a neurodegenerative disease where the level of said pGlu-Aβ peptide in said biological sample is increased compared to the level of said pGlu-Aβ peptide in the healthy control subject.

In a further embodiment, the invention provides a method of monitoring the efficacy of a therapy in a subject having, suspected of having, or being predisposed to a neurodegenerative disease, such as Alzheimer's disease or Mild Cognitive Impairment, comprising determining the level of a pGlu-Aβ peptide in a biological sample from a subject with a method for the detection of a pGlu-Aβ peptide according to the present invention.

In a particular embodiment, said method of diagnosing or said method of monitoring the efficacy of a therapy in a subject having, suspected of having, or being predisposed to a neurodegenerative disease, such as Alzheimer's disease or Mild Cognitive Impairment, comprises the determination of the level of a pGlu-Aβ peptide in a biological sample taken on two or more occasions from a subject.

In one embodiment, the biological sample will be taken on two or more occasions from a test subject. In a further embodiment, the method additionally comprises comparing the level of the pGlu-Aβ peptides present in biological samples taken on two or more occasions from a test subject. In one embodiment, the method additionally comprises comparing the level of the pGlu-Aβ peptides present in a test sample with the amount present in one or more sample(s) taken from said subject prior to commencement of therapy, and/or one or more samples taken from said subject at an earlier stage of therapy. In one embodiment, the method additionally comprises comparing the level of the pGlu-Aβ peptides with one or more controls.

In a further embodiment, said method of diagnosing or said method of monitoring the efficacy of a therapy in a subject further comprises a step, wherein the state of the neurodegenerative disease of the subjects that are donors of the biological samples is characterized in one or more psychometric tests. Suitable psychometric tests for characterization of the state of the neurodegenerative disease of a subject are selected from the DemTect Test, Mini-Mental-State Test, Clock-Drawing Test, ADAS-Cog, Blessed Test, CANTAB, Cognistat, NPI, BEHAVE-AD, CERAD, CSDD, GDS and The 7 Minute Screen.

Suitable treatments of neurodegenerative diseases, such as Alzheimer's diseases and/or Mild Cognitive Impairment, the efficacy of which can be monitored with the methods of the present invention, are treatments that inhibit the formation of the pGlu-residue at the N-terminus of N-terminally truncated Aβ peptides.

Particularly suitable treatments in this regard are inhibitors of the enzyme glutaminyl cyclase. Glutaminyl cyclase has been shown to catalyse the formation of pGlu at the N-terminus of peptides not only from a glutamine residue, but also from a glutamate residue. Accordingly, glutminyl cyclase is responsible for the posttranslational formation of glutamate residues at position 3 or 11 of Aβ peptide to pGlu.

Suitable glutaminyl cyclase inhibitors for the treatment of neurodegenerative diseases, such as Alzheimer's diseases and/or Mild Cognitive Impairment, are for example disclosed in WO 2005/075436, WO 2008/055945, WO 2008/055947, WO 2008/055950, W02008/065141, WO 2008/110523, WO 2008/128981, WO 2008/128982, WO 2008/128983, WO 2008/128984, WO 2008/128985, WO 2008/128986, WO 2008/128987, WO 2010/026212, WO 2010/012828, WO 2011/107530, WO 2011/110613, WO 2011/131748, WO 2012/123563 and WO 2014/140279.

Further suitable treatments of neurodegenerative diseases, such as Alzheimer's diseases and/or Mild Cognitive Impairment, are antibodies, preferably beta-amyloid antibodies, more preferably antibodies that specifically recognize pGlu-Aβ peptides. Suitable pGlu-Aβ antibodies are for example disclosed in WO 2010/009987, WO 2012/123562, U.S. Pat. No. 7,122,374 81, WO 2011/151076, WO 2012/021469; WO 2012/136552 and WO 2010/129276.

In a preferred embodiment, the invention provides a method for monitoring the efficacy of inhibitors of glutaminyl cyclase and/or beta-amyloid antibodies, most preferably antibodies that specifically recognize pGlu-Aβ peptides, in the treatment of neurodegenerative diseases, such as Alzheimer's diseases and/or Mild Cognitive Impairment.

The present method of diagnosis has several advantages over the methods known in the art, i.e. the method of the present invention can be used to detect Alzheimer's disease at an early stage and to differentiate between Alzheimer's disease and other types of dementia in early stages of disease development and progression. One possible early stage is Mild Cognitive Impairment (MCI). It is impossible with the methods currently known in the art to make a clear and reliable diagnosis of early stages of Alzheimer's disease and, in particular, it is impossible to differentiate between the onset of Alzheimer's disease and other forms of dementia in said early stages. This especially applies for patients afflicted with MCI.

In contrast, the methods provided by the present invention are suitable for a differential diagnosis of Alzheimer's disease. In particular, the present invention provides a diagnostic method, wherein the level of pGlu-Aβ peptides can be detected in biological samples obtained from any of the above described subjects in a highly sensitive and reproducible manner. The high sensitivity of the methods of the present invention is achieved by using the detection complex of the invention, the antibodies that are highly specific for the detection of pGlu-Aβ peptides; and the immune-PCR method for the detection and/or quantification of pGlu-Aβ peptides. With the method of the present invention, it is for the first time possible to detect trace amounts or very low amounts of pGlu-Aβ peptides, i.e. down to 4.2 fg/ml, in biological samples such as plasma or CSF. The invention provides a method for the detection of pGlu-Aβ peptides, which is highly sensitive, independently from whether the pGlu-Aβ peptides are present as monomers, in oligomers or bound to proteins in the sample. It is especially possible to detect the occurrence of pGlu-Aβ peptides in a biological sample already closely to or even prior to the onset of Alzheimer's diseases.

The method of the present invention makes it possible for the first time to detect and quantify pGlu-Aβ peptides, in particular those of SEQ ID NOs: 26-37, preferably of SEQ ID NOs: 26-31 and even preferably of SEQ ID NOs: 32-37; or fragments or functional variants thereof, in a highly sensitive manner. In particular, the present invention provides pGlu-Aβ peptides as a biomarker biological fluids, such as plasma or CSF, which is suitable for a differential diagnosis of Alzheimer's disease, in particular in the early stages of the disease.

Therefore, in one embodiment, the invention is directed to the use of the method of determining pGlu-Aβ peptides for the diagnosis of Alzheimer's disease, such as the differential diagnosis of Alzheimer's disease, in particular in the early stages of the disease. Suitably, the early stage of Alzheimer's disease is Mild Cognitive impairment.

In a further embodiment, the invention is directed to the use of the pGlu-Aβ peptides for the diagnosis of Alzheimer's diseases, such as the differential diagnosis of Alzheimer's disease, in particular in the early stages of the disease. Suitably, the early stage of Alzheimer's disease is Mild Cognitive impairment.

In particular, the pGlu-Aβ peptides, which shall be used for diagnosis of Alzheimer's disease, are detected and quantified with a method according to the present invention.

### Deposits of Biological Material

The monoclonal antibodies expressing hybridoma cell lines 5-5-6, 6-1-6, 17-4-3, and 24-2-3 have been deposited in accordance with the Budapest Treaty and are available at the Deutsche Sammlung für Mikroorganismen und Zellkulturen (German Collection of Microorganisms and Cell Cultures) GmbH, DSMZ, Inhoffenstrasse 7B, 38124 Braunschweig,

Germany, with a deposit date of Jun. 17, 2008, and with the respective deposit numbers:
(clone 5-5-6): DSM ACC2923
(clone 6-1-6): DSM ACC2924
(clone 17-4-3): DSM ACC2925
(clone 24-2-3): DSM ACC2926.

The monoclonal antibody expressing hybridoma cell line 13-11-6 has been deposited in accordance with the Budapest Treaty and is available at the Deutsche Sammlung für Mikroorganismen und Zellkulturen (German Collection of Microorganisms and Cell Cultures) GmbH, DSMZ, Inhoffenstrasse 7B, 38124 Braunschweig, Germany, with a deposit date of Dec. 14, 2010, and with the deposit number:
(clone 13-11-6): DSM ACC 3100.

The present invention is further described by the following examples, which should however by no means be construed to limit the invention in any way; the invention is defined in its scope only by the claims as enclosed herewith.

### EXAMPLES OF THE INVENTION

### Example 1

### Highly Sensitive Detection of pGlu-Aβ Peptide

Microplate modules (Chimera biotec C-001) were coated with 30 μl/well capture antibody (clone 6, 17 or 24, probiodrug) at a concentration of 5 μg/m1 in coating buffer (Chimera biotec C-010). Coating was carried out overnight at 4° C. Subsequently, coated modules were washed with wash buffer A (Chimera Biotec, C-011). All washing steps were carried out according to wash buffer manufacturer's instructions. The washed modules were incubated with 30 μl/well sample material, consisting of artificial CSF (Chimera biotec,) spiked with pGlu-Aβ (3-40) or (3-42) (Probiodrug), at different concentration levels and diluted 1+9 in sample dilution buffer (SDB-9100, Chimera Biotec). Incubation was carried out for 45 min at room temperature, followed by a washing step with wash buffer B (Chimera Biotec, C-012). Subsequently, wells were incubated with 30 μl/well biotinylated detection antibody (clone 17 or clone 24, Probiodrug) in a concentration of 0.2 μg/m1 in antibody dilution buffer (SDB-6000, Chimera Biotec). Incubation was carried out for 45 min at room temperature, followed by a washing step with wash buffer B. Subsequently, wells were incubated with 30 μl/well CHI—BIO biotin-binding detection conjugate containing DNA-marker (Chimera biotec,), applied in 1:200 dilution in conjugate dilution buffer (CDB-b, Chimera biotec) for 30 min at room temperature. Following a final washing step with buffer B and buffer A, 30 μl/well PCR-mastermix (Chimera Biotec, C-022) corresponding to the DNA-marker in CHI—BIO are added to each well. The microplate is sealed with PCR-foil (Chimera biotec, C-069) and DNA-amplification & data read-out is carried out according to manufacturer's instructions by application of an lmperacer® workstation (Chimera Biotec 25-002).

### Results

TABLE 1
Quantification of pGlu-Aβ(3-40) with
capture antibody clone 17 or clone 24

Calculated conc [pg/ml]

| | | |
|---|---|---|
| nominal | Capture: | % RE |

| [pg/ml] | Clone 17 | Clone 24 | Clone 17 | Clone 24 |
|---|---|---|---|---|
| 100000 | 112278.5 | 102939.9 | 12 | 3 |
| 10000 | 9516.6 | 9619.4 | 5 | 4 |
| 1000 | 1075.8 | 1169.0 | 8 | 17 |
| 100 | 33.3 | 77.7 | 67 | 22 |
| 10 | 51.1 | 10.5 | 411 | 5 |

TABLE 2
Quantification of pGlu-Aβ(3-42) with capture antibody clone 6

| nominal | calc. Conc. | |
|---|---|---|
| [pg/ml] | [pg/ml] | % RE |
| 1 | 1.1 | 8.9 |
| 10 | 8.3 | 16.7 |
| 100 | 114.2 | 14.2 |
| 1000 | 958.4 | 4.2 |
| 10000 | 9542.0 | 4.6 |
| 100000 | 115430.7 | 15.4 |
| 1000000 | 872503.9 | 12.7 |

### Example 2

### Application of an Antibody—DNA Detection Complex for the Detection of pGlu-Aβ Peptides in a Biological Sample

Microplate modules (Chimera biotec C-001) were coated with 30 μl/well capture antibody (clone 24, Probiodrug) at a concentration of 5 μg/m1 in coating buffer (Chimera biotec C-010). Coating was carried out overnight at 4° C. Subsequently, coated modules were washed with wash buffer A (Chimera Biotec, C-011). All washing steps were carried out according to wash buffer manufacturer's instructions. The washed modules were incubated with 30 μl/well sample material, consisting of artificial CSF (Chimera biotec) spiked with pGlu-Aβ (1+1 mixture of 40 & 42, Probiodrug) at different concentration levels as reference standards and individual CSF for analysis. The sample material was additionally mixed 1+0.03 (one part sample+0.03 part reagent) with an antibody-DNA detection complex (CHI-pGlu, Chimera Biotec, synthesized from clone 17-24, Probiodrug) at sub μg/m1 in artificial CSF (Chimera biotec). Pre-incubation of samples and detection complex was carried out overnight at 4° C. in vials previous to incubation on capture-coated modules; subsequent incubation on capture-coated wells was carried out for 60 min at room temperature, respectively. Following a final washing step with buffer B and buffer A, 30 μl/well PCR-mastermix (Chimera Biotec, C-022) corresponding to the

DNA-marker in CHI—BIO are added to each well. The microplate is sealed with PCR-foil (Chimera biotec, C-069) and DNA-amplification & data read-out is carried out according to manufacturer's instructions by application of an Imperacer® workstation (Chimera Biotec 25-002).

### Results:

TABLE 3
Standards

| nominal | Measured | calc. Conc. | Accuracy | Precision |
|---|---|---|---|---|
| [pg/ml] | delta Ct | [pg/ml] | % RE | % CV |
| 0.046 | 11.79 | 0.036 | 22.11 | 2.4 |
| 0.137 | 12.85 | 0.168 | 22.39 | 13.6 |
| 0.412 | 13.75 | 0.487 | 18.28 | 62.8 |
| 1.235 | 14.51 | 1.065 | 13.79 | 10.3 |
| 3.703 | 15.91 | 3.755 | 1.40 | 13.8 |
| 11.11 | 17.28 | 10.96 | 1.33 | 5.3 |
| 33.33 | 18.81 | 32.23 | 3.31 | 12.1 |
| 100 | 20.60 | 103.2 | 3.19 | 21.2 |

TABLE 4
Individuals

Calculated Concentration

| # | [pg/ml] | CV % |
|---|---|---|
| I | 0.2 | 31.2 |
| II | 1.0 | 9.3 |
| III | 1.9 | 1.4 |
| IV | 2.9 | 10.3 |

### Example 4

### Detection of pGlu3-Aβ Peptides with Pan-Specific anti-β-amyloid Antibodies

The assay protocol of example 3 was repeated with the following modifications:
a) pan-specific antibodies 6E10, BAM90.1 (epitope aa 13-28) or 12F4(specific for Aβ42

C-terminus) were used as detection antibodies in the detection antibody-DNA-conjugate (ADC); and
b) pGlu3-Aβ peptide specific monoclonal antibody clone 6-1-6, clone 17-4-3 or clone 24-2-3 were used as capture antibody for coating the microplate modules.

The preparation of the antibody-DNA-conjugate (ADC) and the detection of pGlu3-Aβ40 and pGlu3-Aβ42 was performed as described in examples 1, 2 and 3.

300 pg/ml of pGlu3-Aβ40 and pGlu3-Aβ42 peptides were detected in the analyzed CSF samples. Antibody clone 6E10 as detection antibody revealed best performance with pGlu3-Aβ peptide specific monoclonal antibody clone 6-1-6 or clone 24-2-3 as capture antibody (see FIG. 1).

### Example 4

### Preparation of Antibody—DNA Detection Complexes for Detection of pGlu-Aβ Peptides

30 μI of a 2.11 pmol/ml solution of 169 bp bis-biotinylated DNA (DNA-marker “1” (SEQ ID NO: 60); 63.3. pmol) were incubated for 30 min at RT with 3.24 μI of a 19.5 pmol/μl solution of recombinant STV (streptavidin, IBA) to form a STV-DNA conjugate (“SDC”). 30 μI of this SDC were mixed with 30 μI of a 500 μg/mI solution of a biotinylated anti-pGlu3-Aβ detection antibody selected from clones 6, 17 and 24 and incubated for 60 min at RT/orbital shaking. The antibody-DNA-STV conjugate was purified by FPLC (Superdex 200) and the 1 ml product fraction was mixed with 2 ml NaCl solution (300 mM) for a final solution of 10.5 pmol/ml detection antibody-DNA-conjugate (“ADC”) (cf. Niemeyer et al., (1999). Nucleic Acids Res 27(23): 4553-61).

### Example 5

### Validation of the pGlu-Aβ Assay

Standard curve and quality controls (QCs) samples with concentrations of pGlu3-Aβ(40/42) of SEQ ID NOs: 28 and 30 were prepared and evaluated according to Table 5. The “low series” contained pGlu3-Aβ(40/42) SEQ ID NOs: 28 and 30 in the range from 4.2 fg/ml up to 9 pg/ml in artificial human CFS. The “high series” contained pGlu3-Aβ(40/42) SEQ ID NOs: 28 and 30 in the range from 78 fg/ml up to 27 pg/ml in artificial human CSF.

The artificial human CSF consists of:
NaCl: 125 mM
KCI: 2.5 mM
MgCI×6 H₂O: 1 mM
NaH₂PO₄: 1.25 mM
CaCl₂×2 H₂O: 2.0 mM
NaHCO₃: 25 mM
Glucose: 25 mM
pH adjusted with NaOH to 7.3
Human serum albumine: 0.3 g/I

Detection of pGlu3-Aβ(40/42) of SEQ ID NOs: 28 and 30 was performed as described in Example 3.

Acceptance criteria for precision (% CV) and accuracy (% RE) for different series (“high” and “low”) of standards and QCs were <20% for the lower limit of quantitation (LLOQ) and <25% for the upper limit of quantitation (ULOQ).

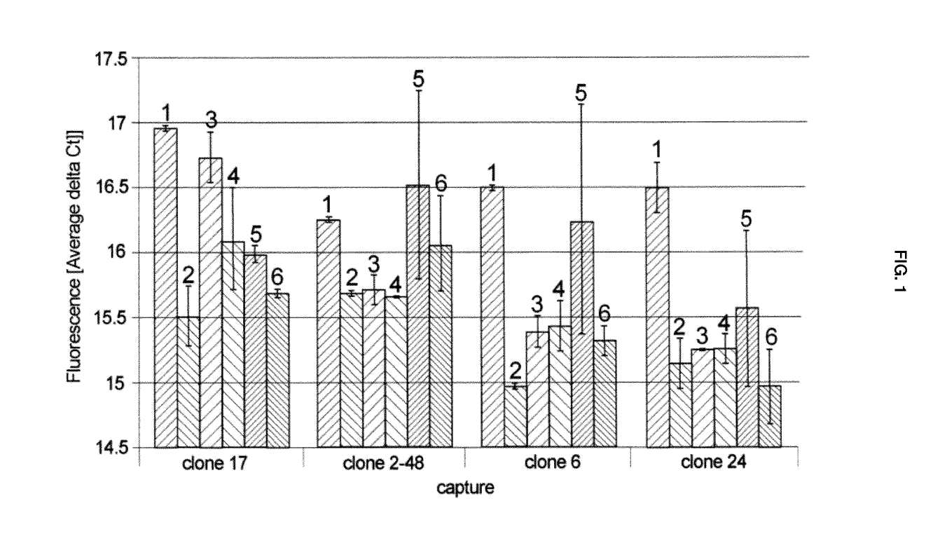

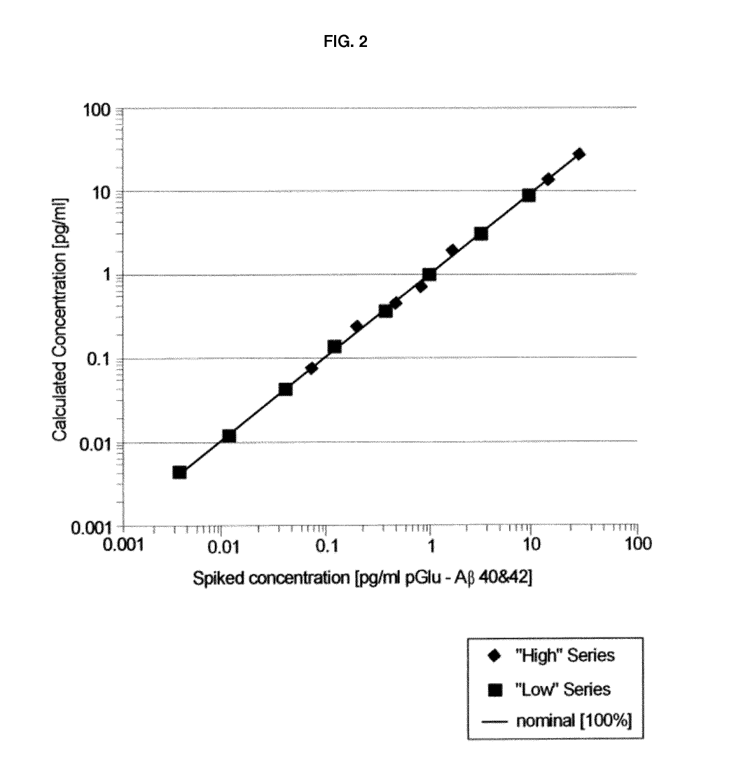

FIG. 2 shows the recovery plot for high and low concentration of standards. It can be seen from FIG. 2 that the developed method allows the detection and quantitation of pGlu-Aβ peptides over a broad linear range of the calibration curve with high precision and accuracy. The method is very sensitive and allows the quantitation of pGlu-Aβ peptides down to 4.2 fg/ml.

TABLE 5
Inter-Assay Average: Precision (% CV) and Accuracy (% RE) for
“high” and “low” standards and QCs

| pg/ml | “high series” | | pg/ml | “low series” | |
|---|---|---|---|---|---|
| [nominal] | % CV | % RE | [nominal] | % CV | % RE |
| Standards | | | Standards | | |
| 27 | 1.18 | 0.8 | 9 | 3.10 | 1.5 |
| 13.5 | 0.94 | 1.6 | 3 | 0.08 | 2.3 |
| 6.75 | 1.29 | 0.7 | 1 | 5.24 | 2.6 |
| 3.375 | 7.06 | 5.8 | 0.34 | 11.10 | 1.8 |
| 1.6875 | 2.76 | 12.0 | 0.12 | 5.92 | 11.2 |
| 0.84375 | 7.34 | 9.0 | 0.042 | 21.55 | 7.8 |
| 0.421875 | 5.14 | 1.5 | 0.0124 | 0.66 | 8.3 |
| 0.210938 | 16.14 | 6.5 | 0.0042 | 17.71 | 8.3 |
| 0.078125 | 6.80 | 2.3 | | | |
| QCs | | | QCs | | |
| 10 | 12.20 | 8.6 | 3.34 | 0.64 | 11.7 |
| 5 | 30.03 | 1.5 | 1.12 | 11.21 | 8.3 |
| 2.5 | 18.95 | 6.5 | 0.38 | 26.29 | 16.10.2014 |
| 1.25 | 1.52 | 3.0 | 0.13 | 12.35 | 0.7 |
| 0.625 | 18.71 | 3.2 | 0.0138 | 4.64 | 26.2 |
| 0.3125 | 17.59 | 5.7 | 0.0046 | 5.52 | 22.0 |
| 0.15625 | 2.44 | 4.7 | | | |

### Example 5

### Determination of pGlu3-Aβ40 (SEQ ID NO: 28) and pGlu3-Aβ42 (SEQ ID NO: 30) in CSF Samples

CSF samples were obtained from patients with a clinical diagnosis of AD and healthy controls according to standard procedures.

A standard curve and quality control samples (QCs) were generated and used as described in Example 5. Acceptance criteria for precision (% CV) and accuracy (% RE) for the CSF samples and QCs were <20% for the lower limit of quantitation (LLOQ) and <25% for the upper limit of quantitation (ULOQ). Individual CSF samples (see sample # in Table 6) were measured as described in Example 3. The pGlu3-Aβ concentration was calculated based on the standard curve.

Table 6 shows the results of the quantitation of pGlu3-Aβ40 (SEQ ID NO: 28) and pGlu3-Aβ42) (SEQ ID NO: 30). The values for the pGlu-Aβ concentration represent the concentration of pGlu3-Aβ40 (SEQ ID NO: 28) and pGlu3-Aβ42 (SEQ ID NO: 30) as a sum parameter.

TABLE 6
Analyzed sample concentration (pGlu3-Aβ40 (SEQ ID NO: 28) and
pGlu3-Aβ42 (SEQ ID NO: 30) in pg/ml) and precision (% CV)

| | pGlu-Aβ concentration | |
|---|---|---|
| Sample # | [pg/ml] | % CV |
| 1 | 0.3 | 26.5 |
| 2 | 0.7 | 21.1 |
| 3 | 0.9 | 20.4 |
| 4 | 1.5 | 1.4 |
| 5 | 1.5 | 22.0 |
| 6 | 1.6 | 29.5 |
| 7 | 5.0 | 1.1 |
| 8 | 5.1 | 8.6 |
| 9 | 6.8 | 22.4 |

The precision (% CV) is used as an acceptance criterion for biomarkers. The precision threshold for a biomarker to accepted is % CV <30%. The results in Table 6 show that the precision (% CV) in all measurements was <30% and therefore meet the precision acceptance criterion for biomarkers.

### Example 7

### Psychometric Tests for Identification of Subjects Suffering from a Neurodegenerative Disease

### 7.1 Materials and Methods

7.1.1 Patients and healthy controls

Patients with a clinical diagnosis of AD and healthy controls were recruited through a CRO. In a prestudy examination the neuropsychological functions of all participants of the study were tested by several psychometric tests (DemTect, Mini-Mental-State Test, Clock-drawing test).

### DemTect Test

The DemTect scale is a brief screening for dementia comprising five short subtests (10-word list repetition, number transcoding, semantic word fluency task, backward digit span, delayed word list recall) (Kessler et al., 2000). The raw scores are transformed to give age- and education-independent scores, classified as ‘suspected dementia’ (score ≦8), ‘mild cognitive impairment’ (score 9-12), and ‘appropriate for age’ (score 13-18).

### MMSE

The Mini-Mental State Examination (MMSE) or Folstein test is a brief 30-point questionnaire test that is used to assess cognition (see Table 4). It is commonly used in medicine to screen for dementia. In the time span of about 10 minutes it samples various functions including arithmetic, memory and orientation. It was introduced by Folstein et al., 1975, and is widely used with small modifications.

The MMSE includes simple questions and problems in a number of areas: the time and place of the test, repeating lists of words arithmetic, language use and comprehension, and basic motor skills. For example, one question asks to copy drawing of two pentagons (see next table). Any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing.

### Clock-Drawing Test

Scoring of the clocks was based on a modification of the scale used by Shulmann et al., 1986. All circles were pre-drawn and the instruction to subjects was to “set the time 10 after 11”. The scoring system (see Table 5) ranges in scores from 1 to 6 with higher scores reflecting a greater number of errors and more impairment. This scoring system is empirically derived and modified on the basis of clinical practice. Of necessity, it leaves considerable scope for individual judgment, but it is simple enough to have a high level of interrater reliability. Our study lends itself to the analysis of the three major components. These include cross-sectional comparisons of the clock-drawing test with other measures of cognitive function; a longitudinal description of the clock-drawing test over time, and the relationship between deterioration on the clock-drawing test and the decisions to institutionalize.

TABLE 7
Mini-Mental State Examination

| | | Max | |
|---|---|---|---|
| Section | Questions | Points | Score |
| 1) Orienta- | a) Can you tell me today's | 5 | |
| tion | (date)/(month)/(year)? | | |
| | Which day is it today? | | |
| | Can you tell me which (season) it is? | | |
| | b) What town/city are we in? | 5 | |
| | What is the (county)! (country)? | | |
| | What (building) are we in and on | | |
| | what (floor) | | |
| 2) Registra- | I should like to test your memory. | 3 | |
| tion | (name three common objects: “ball, car, | | |
| | man”) | | |
| | Can you repeat the words I said? | | |
| | (1 point per word) | | |
| | (repeat up to 6 trials until all three are | | |
| | remembered) | | |
| 3) Attention | a) From 100 keep subtracting 7 and given | 5 | |
| and | each answer. Stop after 5 answers. | | |
| Calculation | (93-86-79-72-65) | | |
| | Alternatively: | | |
| | b) Spell the word “World” backwards. | | |
| | (D_L_R_O_W) | | |
| 4) Recall | What were the three words I asked you to | 3 | |
| | say earlier? | | |
| | (skip this test if all of these objects were | | |
| | not remembered during the registration | | |
| | test) | | |
| 5) Language | Name the following objects (show a watch) | 2 | |
| Naming | and (show a pencil) | | |
| Repeating | Repeat the following: “no if, ands or buts” | 1 | |
| 6) Reading | (show card or write: “Close your Eyes”) | 1 | |
| | Read this sentence and do what it says. | | |
| Writing | Now can you write a short sentence for me? | 1 | |
| 7) Three | (present paper) | 3 | |
| stage | Take this paper in your left (or right) hand, | | |
| command | fold it in half, and place it on the floor. | | |
| 8) Con- | Will you copy this drawing please? | 1 | |
| struction | | | |
| | 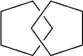 | | |
| Total score | | 30 | |

After prestudy examination the study started 2 weeks later with blood withdrawal from all participants. Over one year with an interval of 3 months all participants had visited the center for the psychometric tests and blood samples withdrawal. The study was approved by the Ethics Committee of the “Ärztekammer Sachsen-Anhalt”. All patients (or their nearest relatives) and controls gave informed consent to participate in the study.

### 6.1.2 Biological Samples

For the analysis of the pGlu-Aβ concentration in humans all of the following body fluids can be used: blood, cerebrospinal fluid, urine, lymph, saliva, sweat, pleura fluid, synovial fluid, aqueous fluid, tear fluid, bile and pancreas secretion.

The novel method was established with CSF samples and can be further used for blood, brain extract and urine samples, followed by all other human body fluids.

CSF samples for the determination of AD biomarkers were collected into three polypropylene tubes:
1. containing potassium-EDTA (Sarstedt Monovette, 02.1066.001) for EDTA plasma
2. containing Li-heparine (Sartstedt Monovette, 02.1065.001) for heparine plasma
3. blank (Sarstedt Monovette, 02.1063.001) for serum

All samples were collected by venous puncture or by repeated withdrawal out of an inserted forearm vein indwelling cannula. Blood was collected according to the time schedule (as described in section 1.1 above). It was centrifuged at 1550 g (3000 rpm) for 10 min at 4° C. to provide plasma. Plasma or serum was pipetted off, filled in one 5 ml polypropylene cryo-tube (Carl-Roth, E295.1) and stored frozen at −80° C. Samples were centrifuged within one hour after blood withdrawal. The appropriate labelling of the plasma or serum tubes according to the study protocol was duty of the CRO.

### 7.2 Results

### 7.2.1 Demographic Characteristics

Overall 45 persons have participated in the study, 30 healthy controls and 15 AD patients. To observe possible influences of age on plasma Aβ, control persons were selected over a wide range of age and subclassified into three groups, Group I contains age of 18 to 30, Group II from 31 to 45 and Group III from 46 to 65. The demographic characteristics are shown in Table 9.

TABLE 9
Demographic Characteristics

| | Healthy controls | | | |
|---|---|---|---|---|
| | Group I | Group II | Group III | |
| | (18-30) | (31-45) | (46-65) | AD patients |
| No. | 10 | 10 | 10 | 15 |
| Age at baseline | 25.8 ± 2.9 | 38.4 ± 4.7 | 54 ± 6.9 | 79.13 ± 7.09 |
| (mean ± SDEV), | | | | |
| Height, cm | 175.5 ± 11.6 | 175.1 ± 7.2 | 167.5 ± 10.9 | 168.4 ± 10.34 |
| (mean ± SDEV) | | | | |
| Weight, kg | 71.33 ± 11.8 | 71.36 ± 13.5 | 75.81 ± 13.3 | 72.00 ± 12.31 |
| (mean ± SDEV) | | | | |
| Sex (% women) | 50 | 50 | 50 | 40 |

### 2.2 Psychometric Tests

For evaluation of the neuropsychological functions all participants have performed the DemTect, Mini-Mental-State Test and Clock-Drawing test. These tests have been made in prestudy, 3 month, 6 month, 9 month and 12 month after the start of the study.

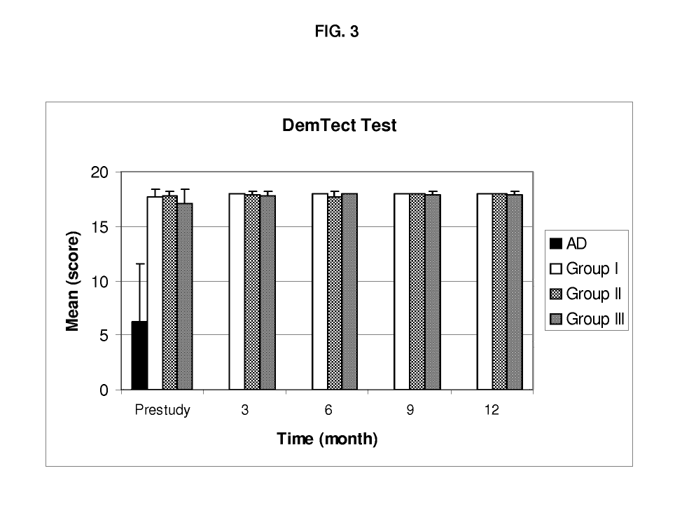

DemTect Test The raw scores are transformed to give age- and education-independent scores, classified as ‘suspected dementia’ (score ≦8), ‘mild cognitive impairment’ (score 9-12), and ‘appropriate for age’ (score 13-18). The test results for all visits are shown in FIG. 3. The results from FIG. 3 demonstrate that there are clear differences between the three groups of healthy subjects compared with the patients.

### Mini-Mental-State Test

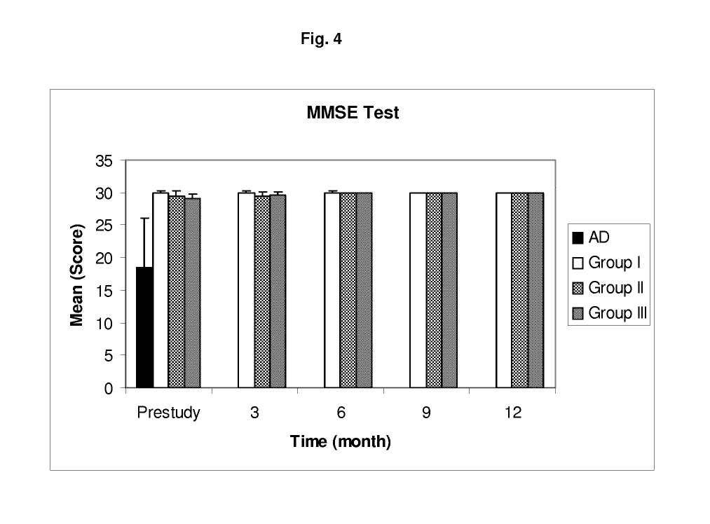

Any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing. The test results are shown in FIG. 4. The results from FIG. 4 demonstrate that there are clear differences between the three groups of healthy subjects compared with the patients.

### Clock-Drawing Test

The scoring system ranges in scores from 1 to 6 with higher scores reflecting a greater number of errors and more impairment. This scoring system is empirically derived and modified on the basis of clinical practice. Of necessity, it leaves considerable scope for individual judgment, but it is simple enough to have a high level of interrater reliability.

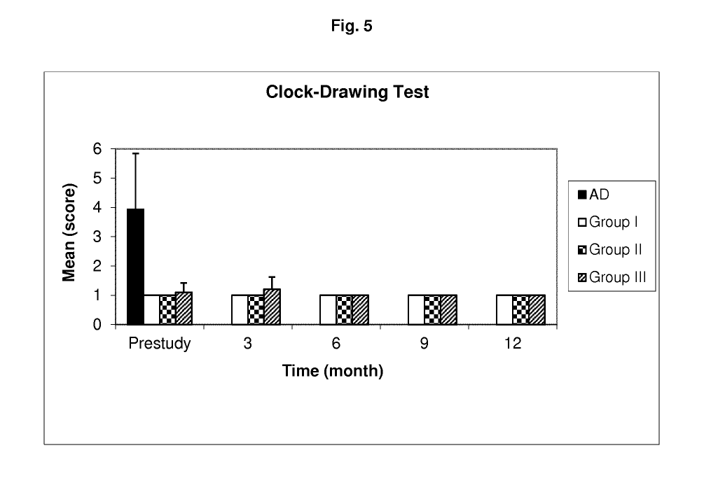

Our study lends itself to the analysis of the three major components. These include cross-sectional comparisons of the clock-drawing test with other measures of cognitive function; a longitudinal description of the clock-drawing test over time, and the relationship between deterioration on the clock-drawing test and the decisions to institutionalize. The test results are shown in FIG. 5. The results from FIG. 5 demonstrate that there are clear differences between the three groups of healthy subjects compared with the patients.

### REFERENCES

Blennow K, de Leon M J, Zetterberg H. Alzheimer's disease. Lancet. 2006 Jul. 29; 368(9533):387-403
Ferri C P, Prince M, Brayne C, Brodaty H, Fratiglioni L, Ganguli M, Hall K, Hasegawa K, Hendrie H, Huang Y, Jorm A, Mathers C, Menezes P R, Rimmer E, Scazufca M Global prevalence of dementia: a Delphi consensus study. Lancet. 2005 Dec. 17; 366 (9503):2112-7
Knopman D S, DeKosky S T, Cummings J L, Chui H, Corey-Bloom J, Relkin N, Small G W, Miller B, Stevens J C. Practice parameter: diagnosis of dementia (an evidence-based review). Report of the Quality Standards Subcommittee of the American Academy of Neurology. Neurology. 2001 May 8; 56(9):1143-53
Waldemar G, Dubois B, Emre M, Georges J, McKeith I G, Rossor M, Scheltens P, Tariska P, Winblad B; EFNS. Recommendations for the diagnosis and management of Alzheimer's disease and other disorders associated with dementia: EFNS guideline. Eur J Neurol. 2007 January; 14(1):e1-26.
Dubois B, Feldman H H, Jacova C, Dekosky S T, Barberger-Gateau P, Cummings J, Delacourte A, Galasko D, Gauthier S, Jicha G, Meguro K, O'brien J, Pasquier F, Robert P, Rossor M, Salloway S, Stern Y, Visser P J, Scheltens P. Research criteria for the diagnosis of Alzheimer's disease: revising the NINCDS-ADRDA criteria. Lancet Neurol. 2007 August; 6(8):734-46.
Jellinger K A. Alzheimer's disease. In: Gilman S, editor. Neurobiologiy of Disease. Amsterdam: Elsevier Academic Press; 2007. p. 69-82
Petersen R C, Smith G E, Waring S C, Ivnik R J, Tangalos E G, Kokmen E. Mild cognitive impairment: clinical characterization and outcome. Arch Neurol. 1999 March; 56(3):303-8
Chertkow H, Massoud F, Nasreddine Z, Belleville S, Joanette Y, Bocti C, Drolet V, Kirk J, Freedman M, Bergman H. Diagnosis and treatment of dementia: 3. Mild cognitive impairment and cognitive impairment without dementia. CMAJ. 2008 May 6; 178(10):1273-85
Scheff S W, Price D A, Schmitt F A, Mufson E J. Hippocampal synaptic loss in early Alzheimer's disease and mild cognitive impairment. Neurobiol Aging. 2006 October; 27(10):1372-84
Markesbery W R, Schmitt F A, Kryscio R J, Davis D G, Smith C D, Wekstein D R. Neuropathologic substrate of mild cognitive impairment. Arch Neurol. 2006 January; 63(1):38-46.
Bouwman F H, Schoonenboom S N, van der Flier W M, van Elk E J, Kok A, Barkhof F, Blankenstein M A, Scheltens P. CSF biomarkers and medial temporal lobe atrophy predict dementia in mild cognitive impairment. Neurobiol Aging. 2007 July; 28(7):1070-4
Saito Y, Murayama S. Neuropathology of mild cognitive impairment. Neuropathology. 2007 December; 27(6):578-84
Jicha G A, Parisi J E, Dickson D W, Johnson K, Cha R, Ivnik R J, Tangalos E G, Boeve B F, Knopman D S, Braak H, Petersen R C. Neuropathologic outcome of mild cognitive impairment following progression to clinical dementia. Arch Neurol. 2006 May; 63(5):674-81
Petersen R C, Parisi J E, Dickson D W, Johnson K A, Knopman D S, Boeve B F, Jicha G A, Ivnik R J, Smith G E, Tangalos E G, Braak H, Kokmen E. Neuropathologic features of amnestic mild cognitive impairment. Arch Neurol. 2006 May; 63(5):665-72
Gauthier S, Reisberg B, Zaudig M, Petersen R C, Ritchie K, Broich K, Belleville S, Brodaty H, Bennett D, Chertkow H, Cummings J L, de Leon M, Feldman H, Ganguli M, Hampel H, Scheltens P, Tierney M C, Whitehouse P, Winblad B; International Psychogeriatric Association Expert Conference on mild cognitive impairment. Mild cognitive impairment. Lancet. 2006 Apr. 15; 367(9518):1262-70
Fischer P, Jungwirth S, Zehetmayer S, Weissgram S, Hoenigschnabl S, Gelpi E, Krampla W, Tragl K H. Conversion from subtypes of mild cognitive impairment to Alzheimer dementia. Neurology. 2007 Jan. 23; 68(4):288-91
Devanand D P, Pradhaban G, Liu X, Khandji A, De Santi S, Segal S, Rusinek H, Pelton G H, Honig L S, Mayeux R, Stern Y, Tabert M H, de Leon M J. Hippocampal and entorhinal atrophy in mild cognitive impairment: prediction of Alzheimer disease. Neurology. 2007 Mar. 13; 68(11):828-36
Rossi R, Geroldi C, Bresciani L, Testa C, Binetti G, Zanetti O, Frisoni G B. Clinical and neuropsychological features associated with structural imaging patterns in patients with mild cognitive impairment. Dement Geriatr Cogn Disord. 2007; 23(3):175-83
Whitwell J L, Petersen R C, Negash S, Weigand S D, Kantarci K, Ivnik R J, Knopman D S, Boeve B F, Smith G E, Jack C R Jr. Patterns of atrophy differ among specific subtypes of mild cognitive impairment. Arch Neurol. 2007 August; 64(8):1130-8
Panza F, Capurso C, D'Introno A, Colacicco A M, Capurso A, Solfrizzi V. Heterogeneity of mild cognitive impairment and other predementia syndromes in progression to dementia. Neurobiol Aging. 2007 October; 28(10):1631-2; discussion 1633-4
Hyman S E. Can neuroscience be integrated into the DSM-V? Nat Rev Neurosci. 2007 September; 8(9):725-32
Blennow K. Cerebrospinal fluid protein biomarkers for Alzheimer's disease. NeuroRx. 2004 April; 1(2):213-25
Blennow K. CSF biomarkers for Alzheimer's disease: use in early diagnosis and evaluation of drug treatment. Expert Rev Mol Diagn. 2005 September; 5(5):661-72
Hampel H, Buerger K. Biomarkers in blood and cerebrospinal fluid. In: Herholz K, Morris C, Perani D, editors. The Dementias: Early Diagnosis and Evaluation. New York: Taylor & Francis; 2006. p. 73-107
Lewczuk P, Kornhuber J, Wiltfang J. The German Competence Net Dementias: standard operating procedures for the neurochemical dementia diagnostics. J Neural Transm. 2006 August; 113(8):1075-80
Irizarry M C. Biomarkers of Alzheimer disease in plasma. NeuroRx. 2004 April; 1(2):226-34
Barnes J, Foster J, Fox N C. Structural magnetic resonace imigaing-derived biomarkers for Alzheimer's disaese. Biomarkers Med. 2007; 1: 79-92
Vemuri P, Gunter J L, Senjem M L, Whitwell J L, Kantarci K, Knopman D S, Boeve B F, Petersen R C, Jack C R Jr. Alzheimer's disease diagnosis in individual subjects using structural MR images: validation studies. Neuroimage. 2008 Feb. 1; 39(3):1186-97
Barkhof F, Polvikoski T M, van Straaten E C, Kalaria R N, Sulkava R, Aronen H J, NiinistöL, Rastas S, Oinas M, Scheltens P, Erkinjuntti T. The significance of medial temporal lobe atrophy: a postmortem MRI study in the very old. Neurology. 2007 Oct. 9; 69(15):1521-7
Mevel K, Desgranges B, Baron J C, Landeau B, De la Sayette V, Viader F, Eustache F, Chételat G. Detecting hippocampal hypometabolism in Mild Cognitive Impairment using automatic voxel-based approaches. Neuroimage. 2007 Aug. 1; 37(1):18-25
Kemppainen N M, Aalto S, Wilson I A, Någren K, Helin S, Brück A, Oikonen V, Kailajärvi M, Scheinin M, Viitanen M, Parkkola R, Rinne J O. PET amyloid ligand [11C]PlB uptake is increased in mild cognitive impairment. Neurology. 2007 May 8; 68(19):1603-6
Klunk W E, Engler H, Nordberg A, Wang Y, Blomqvist G, Holt D P, Bergström M, Savitcheva I, Huang G F, Estrada S, Ausén B, Debnath M L, Barletta J, Price J C, Sandell J, Lopresti B J, Wall A, Koivisto P, Antoni G, Mathis C A, Långström B. Imaging brain amyloid in Alzheimer's disease with Pittsburgh Compound-B. Ann Neurol. 2004 March; 55(3):306-19
Rowe C C, Ng S, Ackermann U, Gong S J, Pike K, Savage G, Cowie T F, Dickinson K L, Maruff P, Darby D, Smith C, Woodward M, Merory J, Tochon-Danguy H, O'Keefe G, Klunk W E, Mathis C A, Price J C, Masters C L, Villemagne V L. Imaging beta-amyloid burden in aging and dementia. Neurology. 2007 May 15; 68(20):1718-25
Pike K E, Savage G, Villemagne V L, Ng S, Moss S A, Maruff P, Mathis C A, Klunk W E, Masters C L, Rowe C C. Beta-amyloid imaging and memory in non-demented individuals: evidence for preclinical Alzheimer's disease. Brain. 2007 November; 130(Pt 11):2837-44
Huang C, Eidelberg D, Habeck C, Moeller J, Svensson L, Tarabula T, Julin P. Imaging markers of mild cognitive impairment: multivariate analysis of CBF SPECT. Neurobiol Aging. 2007 July; 28(7):1062-9
Kantarci K, Weigand S D, Petersen R C, Boeve B F, Knopman D S, Gunter J, Reyes D, Shiung M, O'Brien P C, Smith G E, Ivnik R J, Tangalos E G, Jack C R Jr. Longitudinal 1H MRS changes in mild cognitive impairment and Alzheimer's disease. Neurobiol Aging. 2007 September; 28(9):1330-9
Petrella J R, Wang L, Krishnan S, Slavin M J, Prince S E, Tran T T, Doraiswamy P M. Cortical deactivation in mild cognitive impairment: high-field-strength functional MR imaging. Radiology. 2007 October; 245(1):224-35
Hämäläinen A, Tervo S, Grau-Olivares M, Niskanen E, Pennanen C, Huuskonen J, Kivipelto M, Hänninen T, Tapiola M, Vanhanen M, Hallikainen M, Helkala EL, Nissinen A, Vanninen R, Soininen H. Voxel-based morphometry to detect brain atrophy in progressive mild cognitive impairment. Neuroimage. 2007 Oct. 1; 37(4):1122-31
Kircher T T, Weis S, Freymann K, Erb M, Jessen F, Grodd W, Heun R, Leube D T. Hippocampal activation in patients with mild cognitive impairment is necessary for successful memory encoding. J Neurol Neurosurg Psychiatry. 2007 August; 78(8):812-8
Kropholler M A, Boellaard R, van Berckel B N, Schuitemaker A, Kloet R W, Lubberink M J, Jonker C, Scheltens P, Lammertsma A A. Evaluation of reference regions for (R)-[(11)C]PK11195 studies in Alzheimer's disease and mild cognitive impairment. J Cereb Blood Flow Metab. 2007 December; 27(12):1965-74
Attems J. Sporadic cerebral amyloid angiopathy: pathology, clinical implications, and possible pathomechanisms. Acta Neuropathol. 2005 October; 110(4):345-59
Blennow K, Hampel H. CSF markers for incipient Alzheimer's disease. Lancet Neurol. 2003 October; 2(10):605-13
Hansson O, Zetterberg H, Buchhave P, Londos E, Blennow K, Minthon L. Association between CSF biomarkers and incipient Alzheimer's disease in patients with mild cognitive impairment: a follow-up study. Lancet Neurol. 2006 March; 5(3):228-34
Hansson 0, Zetterberg H, Buchhave P, Andreasson U, Londos E, Minthon L, Blennow K. Prediction of Alzheimer's disease using the CSF Abeta42/Abeta40 ratio in patients with mild cognitive impairment. Dement Geriatr Cogn Disord. 2007; 23(5):316-20
Fagan A M, Mintun M A, Mach R H, Lee S Y, Dence C S, Shah A R, LaRossa G N, Spinner M L, Klunk W E, Mathis C A, DeKosky S T, Morris J C, Holtzman D M. Inverse relation between in vivo amyloid imaging load and cerebrospinal fluid Abeta42 in humans. Ann Neurol. 2006 March; 59(3):512-9
Prince J A, Zetterberg H, Andreasen N, Marcusson J, Blennow K. APOE epsilon4 allele is associated with reduced cerebrospinal fluid levels of Abeta42. Neurology. 2004 Jun. 8; 62(11):2116-8
Strozyk D, Blennow K, White L R, Launer L J. CSF Abeta 42 levels correlate with amyloid-neuropathology in a population-based autopsy study. Neurology. 2003 Feb. 25; 60(4):652-6
Walsh D M, Klyubin I, Shankar G M, Townsend M, Fadeeva J V, Betts V, Podlisny M B, Cleary J P, Ashe K H, Rowan M J, Selkoe D J. The role of cell-derived oligomers of Abeta in Alzheimer's disease and avenues for therapeutic intervention. Biochem Soc Trans. 2005 November; 33(Pt 5):1087-90
Ewers M, Buerger K, Teipel S J, Scheltens P, Schroder J, Zinkowski R P, Bouwman F H, Schonknecht P, Schoonenboom N S, Andreasen N, Wallin A, DeBernardis J F, Kerkman D J, Heindl B, Blennow K, Hampel H. Multicenter assessment of CSF-phosphorylated tau for the prediction of conversion of MCI. Neurology. 2007 Dec. 11; 69(24):2205-12
Fagan A M, Roe C M, Xiong C, Mintun M A, Morris J C, Holtzman D M. Cerebrospinal fluid tau/beta-amyloid(42) ratio as a prediction of cognitive decline in nondemented older adults. Arch Neurol. 2007 March; 64(3):343-9.
Gustafson D R, Skoog I, Rosengren L, Zetterberg H, Blennow K. Cerebrospinal fluid beta-amyloid 1-42 concentration may predict cognitive decline in older women. J Neurol Neurosurg Psychiatry. 2007 May; 78(5):461-4
Li G, Sokal I, Quinn J F, Leverenz J B, Brodey M, Schellenberg G D, Kaye J A, Raskind M A, Zhang J, Peskind E R, Montine T J. CSF tau/Abeta42 ratio for increased risk of mild cognitive impairment: a follow-up study. Neurology. 2007 Aug. 14; 69(7):631-9
Stomrud E, Hansson O, Blennow K, Minthon L, Londos E. Cerebrospinal fluid biomarkers predict decline in subjective cognitive function over 3 years in healthy elderly. Dement Geriatr Cogn Disord. 2007; 24(2):118-24
Hampel H, Teipel S J, Fuchsberger T, Andreasen N, Wiltfang J, Otto M, Shen Y, Dodel R, Du Y, Farlow M, Möller H J, Blennow K, Buerger K. Value of CSF beta-amyloid 1-42 and tau as predictors of Alzheimer's disease in patients with mild cognitive impairment. Mol Psychiatry. 2004 July; 9(7):705-10
Maccioni R B, Lavados M, Guillón M, Mujica C, Bosch R, Farías G, Fuentes P. Anomalously phosphorylated tau and Abeta fragments in the CSF correlates with cognitive impairment in MCI subjects. Neurobiol Aging. 2006 February; 27(2):237-44
Schonknecht P, Pantel J, Kaiser E, Thomann P, Schröder J. Increased tau protein differentiates mild cognitive impairment from geriatric depression and predicts conversion to dementia. Neurosci Lett. 2007 Apr. 6; 416(1):39-42
Clark C M, Xie S, Chittams J, Ewbank D, Peskind E, Galasko D, Morris J C, McKeel D W Jr, Farlow M, Weitlauf S L, Quinn J, Kaye J, Knopman D, Arai H, Doody R S, DeCarli C, Leight S, Lee V M, Trojanowski J Q. Cerebrospinal fluid tau and beta-amyloid: how well do these biomarkers reflect autopsy-confirmed dementia diagnoses? Arch NeuroL 2003 December; 60(12):1696-702
Buerger K, Ewers M, Pirttilä T, Zinkowski R, Alafuzoff I, Teipel S J, DeBernardis J, Kerkman D, McCulloch C, Soininen H, Hampel H. CSF phosphorylated tau protein correlates with neocortical neurofibrillary pathology in Alzheimer's disease. Brain. 2006 November; 129(Pt 11):3035-41
Engelborghs S, Sleegers K, Cras P, Brouwers N, Serneels S, De Leenheir E, Martin J J, Vanmechelen E, Van Broeckhoven C, De Deyn P P. No association of CSF biomarkers with APOEepsilon4, plaque and tangle burden in definite Alzheimer's disease. Brain. 2007 September; 130(Pt 9):2320-6
Buerger K, Alafuzoff I, Ewers M, Pirttilä T, Zinkowski R, Hampel H. No correlation between CSF tau protein phosphorylated at threonine 181 with neocortical neurofibrillary pathology in Alzheimer's disease. Brain. 2007 October; 130(Pt 10):e82
Wiltfang J, Esselmann H, Bibl M, Hüll M, Hampel H, Kessler H, Frölich L, Schröder J, Peters O, Jessen F, Luckhaus C, Perneczky R, Jahn H, Fiszer M, Maler J M, Zimmermann R, Bruckmoser R, Kornhuber J, Lewczuk P. Amyloid beta peptide ratio 42/40 but not A beta 42 correlates with phospho-Tau in patients with low- and high-CSF A beta 40 load. J Neurochem. 2007 May; 101(4):1053-9
Zhong Z, Ewers M, Teipel S, Bürger K, Wallin A, Blennow K, He P, McAllister C, Hampel H, Shen Y. Levels of beta-secretase (BACE1) in cerebrospinal fluid as a predictor of risk in mild cognitive impairment. Arch Gen Psychiatry. 2007 June; 64(6):718-26
(2) Buerger K, Otto M, Teipel S J, Zinkowski R, Blennow K, DeBernardis J, Kerkman D, Schröder J, Schönknecht P, Cepek L, McCulloch C, Möller H J, Wiltfang J, Kretzschmar H, Hampel H. Dissociation between CSF total tau and tau protein phosphorylated at threonine 231 in Creutzfeldt-Jakob disease. Neurobiol Aging. 2006 January; 27(1):10-5
Bibl M, Mollenhauer B, Esselmann H, Schneider M, Lewczuk P, Welge V, Gross M, Falkai P, Kornhuber J, Wiltfang J. Cerebrospinal fluid neurochemical phenotypes in vascular dementias: original data and mini-review. Dement Geriatr Cogn Disord. 2008; 25(3):256-65
McRae A, Martins R N, Fonte J, Kraftsik R, Hirt L, Miklossy J. Cerebrospinal fluid antimicroglial antibodies in Alzheimer disease: a putative marker of an ongoing inflammatory process. Exp Gerontol. 2007 April; 42(4):355-63
Jellinger K A, Janetzky B, Attems J, Kienzl E. Biomarkers for early diagnosis of Alzheimer disease: ‘ALZheimer ASsociated gene’—a new blood biomarker? J Cell Mol Med. 2008 August; 12(4):1094-117
Finehout E J, Franck Z, Choe L H, Relkin N, Lee K H. Cerebrospinal fluid proteomic biomarkers for Alzheimer's disease. Ann Neurol. 2007 February; 61(2):120-9
Castañ E M, Roher A E, Esh CL, Kokjohn T A, Beach T. Comparative proteomics of cerebrospinal fluid in neuropathologically-confirmed Alzheimer's disease and non-demented elderly subjects. Neurol Res. 2006 March; 28(2):155-63
Zhang J, Goodlett D R, Quinn J F, Peskind E, Kaye J A, Zhou Y, Pan C, Y i E, Eng J, Wang Q, Aebersold R H, Montine T J. Quantitative proteomics of cerebrospinal fluid from patients with Alzheimer disease. J Alzheimers Dis. 2005 April; 7(2):125-33
(1)Simonsen A H, McGuire J, Hansson O, Zetterberg H, Podust V N, Davies H A, Waldemar G, Minthon L, Blennow K. Novel panel of cerebrospinal fluid biomarkers for the prediction of progression to Alzheimer dementia in patients with mild cognitive impairment. Arch Neurol. 2007 March; 64(3):366-70
Lescuyer P, Allard L, Zimmermann-lvol C G, Burgess J A, Hughes-Frutiger S, Burkhard PR, Sanchez J C, Hochstrasser D F. Identification of post-mortem cerebrospinal fluid proteins as potential biomarkers of ischemia and neurodegeneration. Proteomics. 2004 August; 4(8):2234-41
Abdi F, Quinn J F, Jankovic J, McIntosh M, Leverenz J B, Peskind E, Nixon R, Nutt J, Chung K, Zabetian C, Samii A, Lin M, Hattan S, Pan C, Wang Y, Jin J, Zhu D, Li G J, Liu Y, Waichunas D, Montine T J, Zhang J. Detection of biomarkers with a multiplex quantitative proteomic platform in cerebrospinal fluid of patients with neurodegenerative disorders. J Alzheimers Dis. 2006 August; 9(3):293-348
Irani D N, Anderson C, Gundry R, Cotter R, Moore S, Kerr D A, McArthur J C, Sacktor N, Pardo C A, Jones M, Calabresi P A, Nath A. Cleavage of cystatin C in the cerebrospinal fluid of patients with multiple sclerosis. Ann Neurol. 2006 February; 59(2):237-47
(2) Hansson S F, Simonsen A H, Zetterberg H, Andersen O, Haghighi S, Fagerberg I, Andréasson U, Westman-Brinkmalm A, Wallin A, Rüetschi U, Blennow K. Cystatin C in cerebrospinal fluid and multiple sclerosis. Ann Neurol. 2007 August; 62(2):193-6
Ravaglia G, Forti P, Maioli F, Chiappelli M, Montesi F, Tumini E, Mariani E, Licastro F, Patterson C. Blood inflammatory markers and risk of dementia: The Conselice Study of Brain Aging. Neurobiol Aging. 2007 December; 28(12):1810-20
Engelhart M J, Geerlings M I, Meijer J, Kiliaan A, Ruitenberg A, van Swieten J C, Stijnen T, Hofman A, Witteman J C, Breteler M M. Inflammatory proteins in plasma and the risk of dementia: the rotterdam study. Arch Neurol. 2004 May; 61(5):668-72
Motta M, Imbesi R, Di Rosa M, Stivala F, Malaguarnera L. Altered plasma cytokine levels in Alzheimer's disease: correlation with the disease progression. Immunol Lett. 2007 Nov. 30; 114(1):46-51
German D C, Gurnani P, Nandi A, Garner H R, Fisher W, Diaz-Arrastia R, O'Suilleabhain P, Rosenblatt KP. Serum biomarkers for Alzheimer's disease: proteomic discovery. Biomed Pharmacother. 2007 August; 61(7):383-9
Ray S, Britschgi M, Herbert C, Takeda-Uchimura Y, Boxer A, Blennow K, Friedman L F, Galasko D R, Jutel M, Karydas A, Kaye J A, Leszek J, Miller B L, Minthon L, Quinn J F, Rabinovici G D, Robinson W H, Sabbagh M N, So Y T, Sparks D L, Tabaton M, Tinklenberg J, Yesavage J A, Tibshirani R, Wyss-Coray T. Classification and prediction of clinical Alzheimer's diagnosis based on plasma signaling proteins. Nat Med. 2007 November; 13(11):1359-62
Graff-Radford N R, Crook J E, Lucas J, Boeve B F, Knopman D S, Ivnik R J, Smith G E, Younkin L H, Petersen R C, Younkin S G. Association of low plasma Abeta42/Abeta40 ratios with increased imminent risk for mild cognitive impairment and Alzheimer disease. Arch Neurol. 2007 March; 64(3):354-62
van Oijen M, Hofman A, Soares H D, Koudstaal P J, Breteler M M. Plasma Abeta (1-40) and Abeta (1-42) and the risk of dementia: a prospective case-cohort study. Lancet Neurol. 2006 August; 5(8):655-60
Sundelöf J, Giedraitis V, Irizarry M C, Sundström J, Ingelsson E, Ronnemaa E, Arnlöv J, Gunnarsson M D, Hyman B T, Basun H, Ingelsson M, Lannfelt L, Kilander L. Plasma beta amyloid and the risk of Alzheimer disease and dementia in elderly men: a prospective, population-based cohort study. Arch Neurol. 2008 February; 65(2):256-63
Song M S, Mook-Jung I, Lee HJ, Min J Y, Park M H. Serum anti-amyloid-beta antibodies and Alzheimer's disease in elderly Korean patients. J Int Med Res. 2007 May-June; 35(3):301-6
Pesaresi M, Lovati C, Bertora P, Mailland E, Galimberti D, Scarpini E, Quadri P, Forloni G, Mariani C. Plasma levels of beta-amyloid (1-42) in Alzheimer's disease and mild cognitive impairment. Neurobiol Aging. 2006 June; 27(6):904-5
Fukumoto H, Tennis M, Locascio J J, Hyman B T, Growdon J H, Irizarry M C. Age but not diagnosis is the main predictor of plasma amyloid beta-protein levels. Arch Neurol. 2003 July; 60(7):958-64
Kosaka T, Imagawa M, Seki K, Arai H, Sasaki H, Tsuji S, Asami-Odaka A, Fukushima T, Imai K, Iwatsubo T. The beta APP717 Alzheimer mutation increases the percentage of plasma amyloid-beta protein ending at A beta42(43). Neurology. 1997 March; 48(3):741-5
Scheuner D, Eckman C, Jensen M, Song X, Citron M, Suzuki N, Bird T D, Hardy J, Hutton M, Kukull W, Larson E, Levy-Lahad E, Viitanen M, Peskind E, Poorkaj P, Schellenberg G, Tanzi R, Wasco W, Lannfelt L, Selkoe D, Younkin S. Secreted amyloid beta-protein similar to that in the senile plaques of Alzheimer's disease is increased in vivo by the presenilin 1 and 2 and APP mutations linked to familial Alzheimer's disease. Nat Med. 1996 August; 2(8):864-70
Sobów T, Flirski M, Kloszewska I, Liberski P P. Plasma levels of alpha beta peptides are altered in amnestic mild cognitive impairment but not in sporadic Alzheimer's disease. Acta Neurobiol Exp (Wars). 2005; 65(2):117-24
Tamaoka A, Fukushima T, Sawamura N, Ishikawa K, Oguni E, Komatsuzaki Y, Shoji S.

Amyloid beta protein in plasma from patients with sporadic Alzheimer's disease. J Neurol Sci. 1996 Sep. 15; 141(1-2):65-8
Vanderstichele H, Van Kerschaver E, Hesse C, Davidsson P, Buyse M A, Andreasen N, Minthon L, Wallin A, Blennow K, Vanmechelen E. Standardization of measurement of beta-amyloid (1-42) in cerebrospinal fluid and plasma. Amyloid. 2000 December; 7(4):245-58
Abdullah L, Paris D, Luis C, Quadros A, Parrish J, Valdes L, Keegan AP, Mathura V, Crawford F, Mullan M. The influence of diagnosis, intra- and inter-person variability on serum and plasma Abeta levels. Neurosci Lett. 2007 Nov. 27; 428(2-3):53-8
Freeman S H, Raju S, Hyman B T, Frosch M P, Irizarry M C. Plasma Abeta levels do not reflect brain Abeta levels. J Neuropathol Exp Neurol. 2007 April; 66(4):264-71
Mehta P D, Pirttila T, Patrick B A, Barshatzky M, Mehta S P. Amyloid beta protein 1-40 and 1-42 levels in matched cerebrospinal fluid and plasma from patients with Alzheimer disease. Neurosci Lett. 2001 May 18; 304(1-2):102-6
Giedraitis V, Sundelöf J, Irizarry M C, Gårevik N, Hyman B T, Wahlund L O, Ingelsson M, Lannfelt L. The normal equilibrium between CSF and plasma amyloid beta levels is disrupted in Alzheimer's disease. Neurosci Lett. 2007 Nov. 12; 427(3):127-31
Blasko I, Jellinger K, Kemmler G, Krampla W, Jungwirth S, Wichart I, Tragl K H, Fischer P. Conversion from cognitive health to mild cognitive impairment and Alzheimer's disease: prediction by plasma amyloid beta 42, medial temporal lobe atrophy and homocysteine. Neurobiol Aging. 2008 January; 29(1):1-11
Mehta P D, Pirttilä T, Mehta S P, Sersen E A, Aisen P S, Wisniewski H M. Plasma and cerebrospinal fluid levels of amyloid beta proteins 1-40 and 1-42 in Alzheimer disease. Arch Neurol. 2000 January; 57(1):100-5
Brettschneider S, Morgenthaler N G, Teipel S J, Fischer-Schulz C, Burger K, Dodel R, Du Y, Möller H J, Bergmann A, Hampel H. Decreased serum amyloid beta (1-42) autoantibody levels in Alzheimer's disease, determined by a newly developed immuno-precipitation assay with radiolabeled amyloid beta (1-42) peptide. Biol Psychiatry. 2005 Apr. 1; 57(7):813-6
Bibl M, Esselmann H, Mollenhauer B, Weniger G, Welge V, Liess M, Lewczuk P, Otto M, Schulz J B, Trenkwalder C, Kornhuber J, Wiltfang J. Blood-based neurochemical diagnosis of vascular dementia: a pilot study. J Neurochem. 2007 October; 103(2):467-74
Walsh D M, Selkoe D J. A beta oligomers—a decade of discovery. J Neurochem. 2007 June; 101(5):1172-84
Lambert M P, Barlow A K, Chromy B A, Edwards C, Freed R, Liosatos M, Morgan T E, Rozovsky I, Trommer B, Viola K L, Wals P, Zhang C, Finch C E, Krafft G A, Klein W L. Diffusible, nonfibrillar ligands derived from Abeta 1-42 are potent central nervous system neurotoxins. Proc Natl Acad Sci USA. 1998 May 26; 95(11):6448-53.
Walsh D M, Klyubin I, Fadeeva J V, Cullen W K, Anwyl R, Wolfe M S, Rowan M J, Selkoe D J. Naturally secreted oligomers of amyloid beta protein potently inhibit hippocampal long-term potentiation in vivo. Nature. 2002 Apr. 4; 416(6880):535-9.
Kayed R, Sokolov Y, Edmonds B, McIntire T M, Milton S C, Hall J E, Glabe C G. Permeabilization of lipid bilayers is a common conformation-dependent activity of soluble amyloid oligomers in protein misfolding diseases. J Biol Chem. 2004 Nov. 5; 279(45):46363-6
Cleary J P, Walsh D M, Hofmeister J J, Shankar G M, Kuskowski M A, Selkoe D J, Ashe K H. Natural oligomers of the amyloid-beta protein specifically disrupt cognitive function. Nat Neurosci. 2005 January; 8(1):79-84.
Pitschke M, Prior R, Haupt M, Riesner D. Detection of single amyloid beta-protein aggregates in the cerebrospinal fluid of Alzheimer's patients by fluorescence correlation spectroscopy. Nat Med. 1998 July; 4(7):832-4
Santos A N, Torkler S, Nowak D, Schlittig C, Goerdes M, Lauber T, Trischmann L, Schaupp M, Penz M, Tiller F W, Böhm G. Detection of amyloid-beta oligomers in human cerebrospinal fluid by flow cytometry and fluorescence resonance energy transfer. J Alzheimers Dis. 2007 March; 11(1):117-25
Klyubin I, Betts V, Welzel A T, Blennow K, Zetterberg H, Wallin A, Lemere C A, Cullen W K, Peng Y, Wisniewski T, Selkoe D J, Anwyl R, Walsh D M, Rowan M J. Amyloid beta protein dimer-containing human CSF disrupts synaptic plasticity: prevention by systemic passive immunization. J Neurosci 2008 Apr. 16; 28(16):4231-7
Roher A E, Baudry J, Chaney M O, Kuo Y M, Stine W B, Emmerling M R. Oligomerizaiton and fibril asssembly of the amyloid-beta protein. Biochim Biophys Acta. 2000 Jul. 26; 1502(1):31-43

Stenh C, Englund H, Lord A, Johansson A S, Almeida C G, Gellerfors P, Greengard P, Gouras G K, Lannfelt L, Nilsson L N. Amyloid-beta oligomers are inefficiently measured by enzyme-linked immunosorbent assay. Ann Neurol. 2005 July; 58(1):147-50.
Englund H, Sehlin D, Johansson A S, Nilsson L N, Gellerfors P, Paulie S, Lannfelt L, Pettersson F E. Sensitive ELISA detection of amyloid-beta protofibrils in biological samples. J Neurochem. 2007 October; 103(1):334-45.
Schupf N, Tang M X, Fukuyama H, Manly J, Andrews H, Mehta P, Ravetch J, Mayeux R. Peripheral Abeta subspecies as risk biomarkers of Alzheimer's disease. Proc Natl Acad Sci U.S.A. 2008 Sep. 16; 105(37):14052-7
Englund H, Degerman Gunnarsson M, Brundin R M, Hedlund M, Kilander L, Lannfelt L, Pettersson F E. Oligomerization partially explains the lowering of Abeta42 in Alzheimer's disease cerebrospinal fluid. Neurodegener Dis. 2009; 6(4):139-47. Epub 2009 Jun. 12
Hansson O, Zetterberg H, Vanmechelen E, Vanderstichele H, Andreasson U, Londos E, Wallin A, Minthon L, Blennow K. Evaluation of plasma Abeta(40) and Abeta(42) as predictors of conversion to Alzheimer's disease in patients with mild cognitive impairment. Neurobiol Aging. 2008 May 16. [Epub ahead of print]
Kessler J, Calabrese P, Kalbe E, Berger F. Ein neues Screening-Verfahren zur Unterstützung der Demenzdiagnostik. Psycho 26 343-347 (2000)
Folstein M F, Folstein S E, McHugh P R. “Mini-mental state”. A practical method for grading the cognitive state of patients for the clinician. J Psychiatr Res. 1975 November; 12(3):189-98
Shulman K I, Shedletsky R, Silver I L. The challenge of time: Clock-drawing and cognitive function in the elderly. Int J Geriatr Psychiatry 1, 135-140 (1986)
Irie K, Murakami K, Masuda Y, Morimoto A, Ohigashi H, Ohashi R, Takegoshi K, Nagao M, Shimizu T, Shirasawa T. Structure of beta-amyloid fibrils and its relevance to their neurotoxicity: implications for the pathogenesis of Alzheimer's disease. J Biosci Bioeng. 2005 May; 99(5):437-47
(2) Simonsen A H, Hansson S F, Ruetschi U, McGuire J, Podust V N, Davies H A, Mehta P, Waldemar G, Zetterberg H, Andreasen N, Wallin A, Blennow K. Amyloid beta 1-40 quantification in CSF: comparison between chromatographic and immunochemical methods. Dement Geriatr Cogn Disord. 2007; 23(4):246-50
Casas C, Sergeant N, Itier J M, Blanchard V, Wirths O, van der Kolk N, Vingtdeux V, van de Steeg E, Ret G, Canton T, Drobecq H, Clark A, Bonici B, Delacourte A, Benavides J, Schmitz C, Tremp G, Bayer TA, Benoit P, Pradier L. Massive CA½ neuronal loss with intraneuronal and N-terminal truncated Abeta42 accumulation in a novel Alzheimer transgenic model. Am J Pathol. 2004 October; 165(4):1289-300
Gelfanova V, Higgs R E, Dean R A, Holtzman D M, Farlow M R, Siemers E R, Boodhoo A, Qian Y W, He X, Jin Z, Fisher D L, Cox K L, Hale J E. Quantitative analysis of amyloid-beta peptides in cerebrospinal fluid using immunoprecipitation and MALDI-Tof mass spectrometry. Brief Funct Genomic Proteomic. 2007 June; 6(2):149-58.
Sergeant N, Bombois S, Ghestem A, Drobecq H, Kostanjevecki V, Missiaen C, Wattez A, David J P, Vanmechelen E, Sergheraert C, Delacourte A. Truncated beta-amyloid peptide species in pre-clinical Alzheimer's disease as new targets for the vaccination approach. J Neurochem. 2003 June; 85(6):1581-91
Xia W, Yang T, Shankar G, Smith I M, Shen Y, Walsh D M, Selkoe DJ. A specific enzyme-linked immunosorbent assay for measuring beta-amyloid protein oligomers in human plasma and brain tissue of patients with Alzheimer disease. Arch Neurol. 2009 February; 66(2):190-9.
El-Agnaf O M, Mahil D S, Patel B P, Austen B M. Oligomerization and toxicity of beta-amyloid-42 implicated in Alzheimer's disease. Biochem Biophys Res Commun. 2000 Jul. 14; 273(3):1003-7
Howlett D R, Perry A E, Godfrey F, Swatton J E, Jennings K H, Spitzfaden C, Wadsworth H, Wood S J, Markwell R E. Inhibition of fibril formation in beta-amyloid peptide by a novel series of benzofurans. Biochem J. 1999 May 15; 340 (Pt 1):283-9

1. A highly sensitive method for the detection of an Aβ target peptide in a biological sample, comprising a capture reagent which is specific for said Aβ target peptide; and an Aβ target peptide detection complex, said method comprising the steps of:
a) contacting a biological sample with said capture reagent and said detection complex; and
b) detecting said Aβ target peptide;
wherein the detection complex comprises an Aβ target peptide specific antibody and a nucleic acid marker.

2. The method of claim 1, wherein said Aβ target peptide is a pGlu-Aβ peptide.

3. The method of claim 1, wherein said detection complex comprises a detection antibody capable of binding a pGlu-Aβ peptide, at least one nucleic acid marker comprising a predetermined nucleotide sequence; and at least one first linker molecule adapted to bind said antibody and the nucleic acid marker.

4. The method according to claim 1, comprising the steps of:
a) contacting the biological sample with the detection complex under conditions allowing the binding of an Aβ target peptide to said detection complex; and
b) subsequently contacting said Aβ target peptide, which is bound to the detection complex, with a capture reagent capable of binding an Aβ target peptide peptide under conditions allowing the binding of said capture reagent to said Aβ target peptide.

5. The method of claim 4, wherein the capture reagent is a capture antibody specific for a pGlu-Aβ peptide.

6. The method according to claim 1, comprising the steps of:
a) contacting a biological sample with at least one detection complex, wherein said detection complex comprises a detection antibody capable of binding an Aβ target peptide, at least one nucleic acid marker comprising a predetermined nucleotide sequence and at least one first linker molecule adapted to bind said antibody and the nucleic acid marker; under conditions allowing the binding of said detection complex to said Aβ target peptide;
b) further contacting said Aβ target peptide, which is bound to the detection complex, with a capture antibody capable of binding said Aβ target peptide under conditions allowing the binding of said capture antibody to said Aβ target peptide; and
c) detecting said Aβ target peptide;
wherein said Aβ target peptide is a pGlu-Aβ peptide; and
wherein at least one of the detection antibody and the capture antibody specifically binds to the pyroglutamate carrying amino terminus of said pGlu-Aβ peptide.

7. The method according to claim 1, wherein both of the detection antibody and the capture antibody specifically bind to the pyroglutamate carrying amino terminus of said pGlu-Aβ peptide.

8. The method according to claim 1, wherein the detection antibody specifically binds to the pyroglutamate carrying amino terminus of said pGlu-Aβ peptide and the capture antibody binds to an epitope sequence of an Aβ peptide other than the pyroglutamate carrying amino terminus.

9. The method according to claim 1, wherein the capture antibody specifically binds to the pyroglutamate carrying amino terminus of said pGlu-Aβ peptide and the detection antibody binds to an epitope sequence of an Aβ peptide other than the pyroglutamate carrying amino terminus.

10. The method according to claim 1, wherein the detection complex is provided in a matrix similar to the biological sample and added directly to the biological sample in a ratio lower than 1+1.

11. The method according to claim 2, wherein pGlu-Aβ peptides are detected as monomers and/or in Aβ peptide oligomers and/or bound to proteins in the biological sample.

12. The method according to claim 2, wherein said pGlu-Aβ is at least one pGlu-Aβ peptide selected from SEQ ID NOs: 26 to 37.

13. The method according to claim 6, wherein the detection antibody and/or the capture antibody specifically binds to the pyroglutamate carrying amino terminus of said at least one pGlu-Aβ peptide selected from SEQ ID NOs: 26 to 31.

14. The method according to claim 13, wherein the detection antibody and/or the capture antibody that specifically binds to the pyroglutamate carrying amino terminus of said at least one pGlu-Aβ peptide of SEQ ID NOs: 26 to 31, is selected from the group consisting of:
pGlu3-Aβ antibody clone 2-48 (monoclonal, mouse); Synaptic Systems,
pGlu3-Aβ antibody (polyclonal, rabbit); Synaptic Systems, Biotrend, IBL,
pGlu3-Aβ antibody clone 8E1 (monoclonal, mouse); Anawa,
pGlu3-Aβ antibody clone 8E1 (monoclonal, mouse); Biotrend,
Anti-Human Amyloidβ (N3pE) Rabbit IgG (polyclonal, rabbit); IBL,
Abeta-pE3 rabbit polyclonal, affinity purified, Synaptic systems,
Anti-Human Aβ N3pE (8E1) Mouse IgG Fab (monoclonal, mouse); IBL,
pGlu3-Aβ antibody clone 337.48 (monoclonal, mouse); Biolegend,
pGlu3-Aβ antibody clone 1-57 (monoclonal, mouse); Synaptic Systems,
pGlu3-Aβ antibody clone 70D7 (monoclonal, mouse); Synaptic Systems, and
oligo pGlu3-Aβ antibody clone 9D5 (monoclonal, mouse); Synaptic Systems.

15. (canceled)

16. The method according to claim 13, wherein the variable part of the light chain of said detection antibody and/or said capture antibody has the nucleotide sequence of SEQ ID NO:
40 or the amino acid sequence of SEQ ID NO: 41, and wherein the variable part of the heavy chain of said detection antibody and/or said capture antibody has the nucleotide sequence of SEQ ID NO: 42, or the amino acid sequence of SEQ ID NO: 43.

17. The method according to claim 13, wherein the variable part of the light chain of said detection antibody and/or said capture antibody has the nucleotide sequence of SEQ ID NO:
44 or the amino acid sequence of SEQ ID NO: 45, and wherein the variable part of the heavy chain of said detection antibody and/or said capture antibody has the nucleotide sequence of SEQ ID NO: 46, or the amino acid sequence of SEQ ID NO: 47.

18. The method according to claim 13, wherein the variable part of the light chain of said detection antibody and/or said capture antibody has the nucleotide sequence of SEQ ID NO:
48 or the amino acid sequence of SEQ ID NO: 49, and wherein the variable part of the heavy chain of said detection antibody and/or said capture antibody has the nucleotide sequence of SEQ ID NO: 50, or the amino acid sequence of SEQ ID NO: 51.

19. The method according to claim 13, wherein the variable part of the light chain of said detection antibody and/or said capture antibody has the nucleotide sequence of SEQ ID NO:
52 or the amino acid sequence of SEQ ID NO: 53, and wherein the variable part of the heavy chain of said detection antibody and/or said capture antibody has the nucleotide sequence of SEQ ID NO: 54, or the amino acid sequence of SEQ ID NO: 55.

20. The method according to claim 6, wherein said detection antibody and/or said capture antibody is a monoclonal antibody produced by a hybridoma cell line selected from the group consisting of:

| | |
|---|---|
| Aβ 5-5-6 | (Deposit No. DSM ACC 2923) |
| Aβ 6-1-6 | (Deposit No. DSM ACC 2924) |
| Aβ 17-4-3 | (Deposit No. DSM ACC 2925) |
| Aβ 24-2-3 | (Deposit No. DSM ACC 2926). |

21. The method according to claim 2, wherein said pGlu-Aβ peptide is at least one pGlu-Aβ peptide of SEQ ID NOs: 32 to 37.

22. The method according to claim 6, wherein the detection antibody and/or the capture antibody that specifically binds to the pyroglutamate carrying amino terminus of said pGlu-Aβ(11-x) peptide pGlu-Aβ peptide of SEQ ID NOs: 32 to 37, is selected from the group consisting of
pGlu11-Aβ antibody clone 173D8, (monoclonal, mouse); Synaptic Systems, and
pGlu11-Aβ antibody (polyclonal rabbit); Synaptic Systems.

23. The method according to claim 6, wherein the detection antibody and/or the capture antibody specifically binds to epitope sequence pGlu-VHH of SEQ ID NO: 39.

24. The method according to claim 6, wherein the variable part of the light chain of said detection antibody and/or said capture antibody has the nucleotide sequence of SEQ ID NO: 56 or the amino acid sequence of SEQ ID NO: 57, and wherein the variable part of the heavy chain of said detection antibody and/or said capture antibody has the nucleotide sequence of SEQ ID NO: 58, or the amino acid sequence of SEQ ID NO: 59.

25. The method according to claim 6, wherein said detection antibody and/or said capture antibody is a monoclonal antibody produced by hybridoma cell line Aβ 13-11-6 (Deposit No. DSM ACC 3100).

26. The method according to claim 6, wherein said detection antibody and/or said capture antibody binds to an epitope of the Aβ peptide other than the pyroglutamate carrying amino terminus of a said pGlu-Aβ peptide.

27. The method according to claim 26, wherein said detection antibody and/or said capture antibody is selected from the group consisting of:
3D6, Epitope:1-5 (Elan Pharmaceuticals, Innogenetics),
pAb-EL16, Epitope: 1-7,
2H4, Epitope: 1-8 (Covance),
1E11, Epitope: 1-8 (Covance),
20.1, Epitope: 1-10 (Covance, Santa Cruz Biotechnology),
Rabbit Anti-Aβ (3 Polyclonal Antibody, Epitope: 1-14 (Abcam),
AB10, Epitope: 1-16 (Chemicon/Upstate—part of Millipore),
82E1, Epitope: 1-16 (IBL),
pAb 1-42, Epitope: 1-11,
NAB228, Epitope: 1-11 (Covance, Sigma-Aldrich, Cell Signaling, Santa Cruz Biotechnology, Zymed/Invitrogen),
DE2, Epitope: 1-16 (Chemicon/Upstate—part of Millipore),
DE2B4, Epitope: 1-17 (Novus Biologicals, Abcam, Accurate, AbD Serotec),
6E10, Epitope: 1-17 (Signet Covance, Sigma-Aldrich),
10D5, Epitope: 3-7 (Elan Pharmaceuticals),
WO-2, Epitope: 4-10 (The Genetics Company),
1A3, Epitope 5-9 (Abbiotec),
pAb-EL21, Epitope 5-11,
310-03, Epitope 5-16 (Abeam, Santa Cruz Biotechnology),
Chicken Anti-Human Aβ Polyclonal Antibody, Epitope 12-28 (Abeam),
Chicken Anti-Human Aβ Polyclonal Antibody, Epitope 25-35 (Abeam),
Rabbit Anti-Human Aβ Polyclonal Antibody, Epitope: N-terminal (ABR),
Rabbit Anti-Human Aβ Polyclonal Antibody (Anaspec),
12C3, Epitope 10-16 (Abbiotec, Santa Cruz Biotechnology),
16C9, Epitope 10-16 (Abbiotec, Santa Cruz Biotechnology),
19B8, Epitope 9-10 (Abbiotec, Santa Cruz Biotechnology),
pAb-EL26, Epitope: 11-26,
BAM90.1, Epitope: 13-28 (Sigma-Aldrich),
Rabbit Anti-beta-Amyloid (pan) Polyclonal Antibody, Epitope: 15-30 (MBL),
22D12, Epitope: 18-21 (Santa Cruz Biotechnology),
266, Epitope: 16-24 (Elan Pharmaceuticals),
pAb-EL17; Epitope: 15-24,
4G8, Epitope: 17-24 (Covance),
Rabbit Anti-Aβ Polyclonal Antibody, Epitope: 22-35 (Abeam),
G2-10; Epitope: 31-40 (The Genetics Company),
Rabbit Anti-Aβ aa 32-40 Polyclonal Antibody (GenScript Corporation),
EP1876Y, Epitope: x-40 (Novus Biologicals),
G2-11, Epitope: 33-42 (The Genetics Company),
16C11, Epitope: 33-42 (Santa Cruz Biotechnology),
21F12, Epitope: 34-42 (Elan Pharmaceuticals, Innogenetics),
1A10, Epitope: 35-40 (IBL),
D-17 Goat anti-Aβ antibody, Epitope: C-terminal (Santa Cruz Biotechnology),
2C8, Epitope: 1-16 (Accurate),
BAM-10, Epitope: 1-12 (Biotrend, Sigma-Aldrich),
12B2, Epitope: 11-28 (IBL, Biotrend),
6F/3D, Epitope: 8-17 (Accurate),
310-01, Epitope: 10-16, (Accurate),
11A5-B10, Epitop: 34-40, (Millipore),
12F4, Epitope: 36-42, (Millipore, Covance),
9C4, Epitope: 37-43, (Milipore),
7N22, Epitope: 1-20, (Biosource),
11A50-B10, Epitope: 35-40) (Covance),
G2-13, Epitope: C-terminus Aβ42 (Genetics Company),
2B9, Epitope: 1-17 (Santa Cruz), and
9C4, Epitope: 3-8 (Covance).

28. The method according to claim 3, wherein the detection complex further comprises at least one second linker molecule capable of specifically binding the first linker molecule.

29. The method according to claim 2, wherein the detection of said pGlu-Aβ peptide is performed with an immuno-PCR reaction.

30. The method according to claim 2, wherein a mixture of different pGlu-Aβ peptides, is applied as a reference substance for quantification.

31. (canceled)

32. A method of diagnosing or monitoring a neurodegenerative disease, such as Alzheimer's disease and Mild Cognitive Impairment, which comprises determining the level of a pGlu-Aβ peptide in a biological sample from a test subject, comprising the following steps:
i. determining a first level of a pGlu-Aβ peptide in a biological sample from a subject suspected to be afflicted with said neurodegenerative disease with a method according to claim 2;
ii. comparing the first level of the pGlu-Aβ peptide with a second level of said pGlu-Aβ peptide in a healthy control subject; and
iii. diagnosing the subject with a neurodegenerative disease where the level of said pGlu-Aβ peptide in said biological sample is increased compared to the level of said pGlu-Aβ peptide in the healthy control subject.

33. A method of monitoring the efficacy of a therapy in a subject having, suspected of having, or being predisposed to a neurodegenerative disease, such as Alzheimer's disease or Mild Cognitive Impairment, comprising determining determining the level of a pGlu-Aβ peptide in a biological sample from a test subject with a method according to claim 2.

34. The method of diagnosing or monitoring as defined in claim 32, which comprises determining the level of a pGlu-Aβ peptide in a biological sample taken on at least two or more occasions from a test subject.

35. The method according to claim 32, wherein the state of the neurodegenerative disease of the subjects that are donors of the biological samples is characterized in at least one or more psychometric test.

36. The method according to claim 35, wherein said psychometric test is selected from the DemTect Test, Mini-Mental-State Test, Clock-Drawing Test, ADAS-Cog, Blessed Test, CANTAB, Cognistat, NPI, BEHAVE-AD, CERAD, CSDD, GDS and the The 7 Minute Screen.

37. The method or use according to claim 1, wherein the biological sample is selected from the group consisting of tissue, blood, serum, urine, cerebrospinal fluid (CSF), plasma, lymph, saliva, sweat, pleural fluid, synovial fluid, tear fluid, bile and pancreas secretion.

38. A kit for diagnosing a neurodegenerative disease, such as Alzheimer's disease or Mild Cognitive Impairment, which comprises at least one detection complex, at least one capture antibody and instructions for using the kit, wherein said detection complex comprises, essentially consists of or consists of a detection antibody capable of binding an Aβ target peptide, one or more nucleic acid markers comprising a predetermined nucleotide sequence and at least one first linker molecule adapted to bind said antibody and the nucleic acid marker, and wherein at least one of the detection antibody and the capture antibody specifically binds to the pyroglutamate carrying amino terminus of the Aβ target peptide.

39. The kit of claim 38, wherein the detection complex further comprises at least one second linker molecule capable of specifically binding the first linker molecule.

40. A detection complex comprising, a detection antibody capable of binding an Aβ target peptide, at least one or more nucleic acid marker markers comprising a predetermined nucleotide sequence and at least one first linker molecule adapted to bind said antibody and the nucleic acid marker.

41. The detection complex of claim 40, further comprising at least one second linker molecule capable of specifically binding the first linker molecule.

42. The detection detectiondetection complex of claim 40, wherein the detection antibody specifically binds to the pyroglutamate carrying amino terminus of a pGlu-Aβ peptide.
